SEQUENCE LISTING (6 sequences)

SEQ ID NO: 1 - protein, 11 aa, Bacillus anthracis
  minimal (1)..(11): Protective Antigen residues 308-318
EVHASFFDIGG

SEQ ID NO: 2 - protein, 29 aa, Bacillus anthracis
  maximal (1)..(29): Protective Antigen residues 299-327
HTSEVHGNAEVHASFFDIGGSVSAGFSNS

SEQ ID NO: 3 - protein, 16 aa, Bacillus anthracis
  optimal (1)..(16): Protective Antigen residues 304-319
HGNAEVHASFFDIGGS

SEQ ID NO: 4 - protein, 37 aa, Artificial Sequence
  Synthetic Peptide
FNNFTVSFWLRVPKVSASHLEHGNAEVHASFFDIGGS

SEQ ID NO: 5 - protein, 36 aa, Artificial Sequence
  Synthetic Peptide
HGNAEVHASFFDIGGSEYLNKIQNSLSTEWSPCSVT

SEQ ID NO: 6 - protein, 57 aa, Artificial Sequence
  Synthetic Peptide
FNNFTVSFWLRVPKVSASHLEHGNAEVHASFFDIGGSEYLNKIQNSLSTEWSPCSVT

(19) United States
(12) Patent Application Publication
(10) Pub. No.: US 2011/0256172 A1
(43) Pub. Date: Oct. 20, 2011

(54) EPITOPE-TARGETED ANTHRAX VACCINE

(75) Inventors: Kemp B. Cease, Ann Arbor, MI (US); Jon Oscherwitz, Ann Arbor, MI (US)

(73) Assignee: THE REGENTS OF THE UNIVERSITY OF MICHIGAN, Ann Arbor, MI (US)

(21) Appl. No.: 13/124,167

(22) PCT Filed: Oct. 14, 2009
(86) PCT No.: PCT/US2009/060684

§ 371 Date: Jul. 7, 2011

Related U.S. Application Data
(60) Provisional application No. 61/196,054, filed on Oct. 14, 2008.

(51) Int. Cl. A61K 39/07; A61P 37/04; A61P 31/04; C07K 19/00

(52) U.S. Cl. 424/197.11; 530/300; 530/350; 530/317

(57) ABSTRACT

Anthrax vaccine compositions comprise a segment of a PA toxin protein that stimulates a B cell immune response specific for a defined epitope on the protective antigen of B. anthracis, a pharmaceutical excipient and optionally, one or more other protein segments comprising epitopes that augment the B cell response by stimulating a T cell immune response. The pharmaceutical compositions are useful for vaccinating individuals so as to confer protection from disease caused by B. anthracis including anthrax disease resulting from anthrax spore inhalation.

### CROSS-REFERENCE TO RELATED APPLICATIONS

This application is a PCT International Application of U.S. Provisional Application No. 61/196,054, filed on Oct. 14, 2008. The entire disclosure of the above application is incorporated herein by reference.

### GOVERNMENT SUPPORT

This invention was made with government support under grant number U01-AI056580 awarded by the National Institutes of Health. The government has certain rights in the invention.

### FIELD

The present disclosure relates to vaccines for the prevention of disease in mammals, including humans, caused by Bacillus anthracis (anthrax). More particularly, it relates to the prevention of inhalation anthrax resulting from inhalation of spores, as in the event of exposure to such spores dispersed in an attack in which they are employed as a weapon.

### BACKGROUND

This section provides background information related to the present disclosure which is not necessarily prior art.

Bacillus anthracis is a formidable bioterrorism and biowarfare threat for which new vaccines are needed. An ideal lethal-toxin-targeted vaccine should induce rapid and durable toxin-neutralizing antibody responses, do so with a minimum of acute and chronic side effects, and should have favorable production, storage, and administration characteristics. These needs have motivated research on development of improved anthrax vaccines. There is no report, however, of the successful creation of a vaccine for eliciting active immunity specific for a critical neutralizing epitope on protective antigen or any other components of the anthrax toxins. Inhalation anthrax, resulting from an attack with optimally formulated and dispersed B. anthracis spores, represents a grave threat to both military and civilian populations. Though post-event prophylaxis and therapy would play important mitigating and recovery roles, pre-existing vaccine-induced immunity would represent the initial means of preventing morbidity and mortality from such an attack.

The morbidity and mortality of inhalation anthrax in humans is largely a direct result of the elaboration of lethal toxin (LeTx) during the vegetative growth of virulent strains of B. anthracis. Anthrax toxins, LeTx and edema toxin are classic A-B toxins, where lethal factor (LF) and edema factor (EF) represent the active moieties and protective antigen (PA), the binding moiety. PA binds the anthrax toxin receptor, either CMG2 or TEM8, and forms a heptameric pre-pore which binds EF or LF. Under the acidic conditions of the late endosome, EF and LF are transported into the cell cytosol where they exert their enzymatic activities as edema toxin and LeTx, respectively.

PA-specific humoral immunity has been demonstrated to protect animals from experimental challenge with anthrax even in the absence of LF and EF immunity. Animal model studies have shown that Anthrax Vaccine Adsorbed (AVA, BioThrax®, Emergent Biosolutions, Rockville, Md.), the currently licensed anthrax vaccine in the U.S., provides protection by stimulating antibodies against PA, and AVA has been shown to confer a high degree of protection from an inhalation spore challenge in rabbits and primates. However, the multiple injections and yearly boosts required for establishment and maintenance of immunity, and the reactigenicity and potential adverse reactions to AVA, have raised broad concern, and have motivated commitment to the development of next generation anthrax vaccines.

AVA is prepared from a heat-inactivated cell-free preparation from a non-encapsulated strain of B. anthracis. Protective Antigen (PA), the receptor binding component of the anthrax toxin, is a major component of AVA, and the immune response to PA appears to mediate protection. The level of anti-PA antibody and its avidity for PA correlate with activity in toxin neutralization assays (TNAs) in vitro. TNA activity in turn, correlates with in vivo protection from inhalation anthrax following an aerosolized spore challenge in rabbit and primate models. Consequently, the elicitation of PA-specific toxin-neutralizing antibody appears to be critical for the efficacy of AVA and other PA-based vaccines.

The reliability of PA-based vaccine protection in the context of various natural or malicious scenarios may however depend on the specificity repertoire of the anti-PA neutralizing antibodies. Monoclonal antibody studies suggest that this repertoire may be quite limited, possibly focused predominantly on only one or a limited number of neutralizing sites. From a reliability engineering perspective, reliance on a response to a single site for protection would represent a potential single point of failure, and a critical vulnerability in the system. Failure to develop a neutralizing antibody specific for the site would result in failure of vaccine protection for an individual or population. Examples of such failures are evident in immune protection from several pathogens, including hepatitis B virus, rabies virus, respiratory syncytial virus, B. burgdorferi, N. meningitides, HIV, and influenza. In these cases the agent's mutability and transmissibility in nature enables emergence of escape mutations. With anthrax, the threat of vaccine-escape strains derives not only from forces of nature, but from the potential for malicious selection and reengineering in the laboratory.

Most of the efforts to develop new vaccines for anthrax have focused on the elicitation of immunity to PA. Ongoing research is also focused on the design and testing of vaccines targeting antigens other than PA in an effort to broaden the breadth of immunity induced through vaccination. This has been motivated in part by the realization, informed primarily from the analysis of PA-specific monoclonal antibodies in mice and humans, that the antibody specificities responsible for LeTx neutralization may be limited to only a few dominant specificities. These are focused primarily on domains 1′ and 4 of PA63 which are involved in binding to LF and EF, and to the anthrax toxin receptors, respectively. Limited breadth of the neutralizing repertoire induced through PA immunization could leave vaccinated individuals vulnerable to possible maliciously altered or selected B. anthracis strains resistant to the neutralizing specificities.

### SUMMARY

This section provides a general summary of the disclosure, and is not a comprehensive disclosure of its full scope or all of its features.

The present disclosure provides an immunogen useful for inducing an immunostimulatory response against an anthrax infection. The immunogen comprises a loop neutralizing determinant (LND), where the LND can comprise a polypeptide sequence having at least 75% identity to amino acids 304-319 of Bacillus anthracis protective antigen (PA).

Methods for inducing an immunostimulatory response to an anthrax infection in a subject include administering a pharmaceutical composition comprising an immunogen comprising a recombinant polypeptide having a PA toxin loop neutralizing determinant polypeptide sequence and at least one pharmacologically acceptable excipient.

Accordingly, it is an object of the present disclosure to provide synthetic peptide and recombinant protein antigens capable of producing, in vivo, a neutralizing antibody capable of neutralizing protective antigen function.

Another object is to provide synthetic peptide and recombinant protein vaccines which comprise such antigens as active ingredients.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 shows linear peptide and multiple antigenic peptide constructions incorporating B cell epitopes representing the LND. Panel (A) depicts three polypeptide constructs incorporating the B. anthracis PA LND sequence in accordance with the present disclosure. Linear peptide construct 1 is a linear peptide of SEQ ID NO: 4 comprised of the P30 helper T cell epitope from the toxin of Clostridium tetani synthesized colinearly at the N-terminus of a B cell epitope representing the LND of SEQ ID NO: 3. Linear peptide construct 2 is a linear peptide of SEQ ID NO: 5 comprised of the LND segment of SEQ ID NO: 3 synthesized colinearly with the Plasmodium falciparum CS protein T* helper T cell epitope at the C-terminus. Linear peptide construct 3 is a linear peptide of SEQ ID NO: 6 comprised of the LND segment of SEQ ID NO: 3 synthesized colinearly with the P30 and CS protein T* helper T cell epitope at its N- and C-termini, respectively. Panel (B) depicts the polypeptide sequence and arrangement of a multiple antigenic peptide (MAP) construct comprised of four copies of the LND peptide of SEQ ID NO: 4 synthesized from the α- and ε-amino groups of a branching lysine core. Panel (C) depicts an illustration of a linearized polypeptide including a maltose binding protein (MBP) fused in frame with a S. mansoni p38-P4 polypeptide and a B. anthracis PA LND sequence in accordance with the present disclosure.

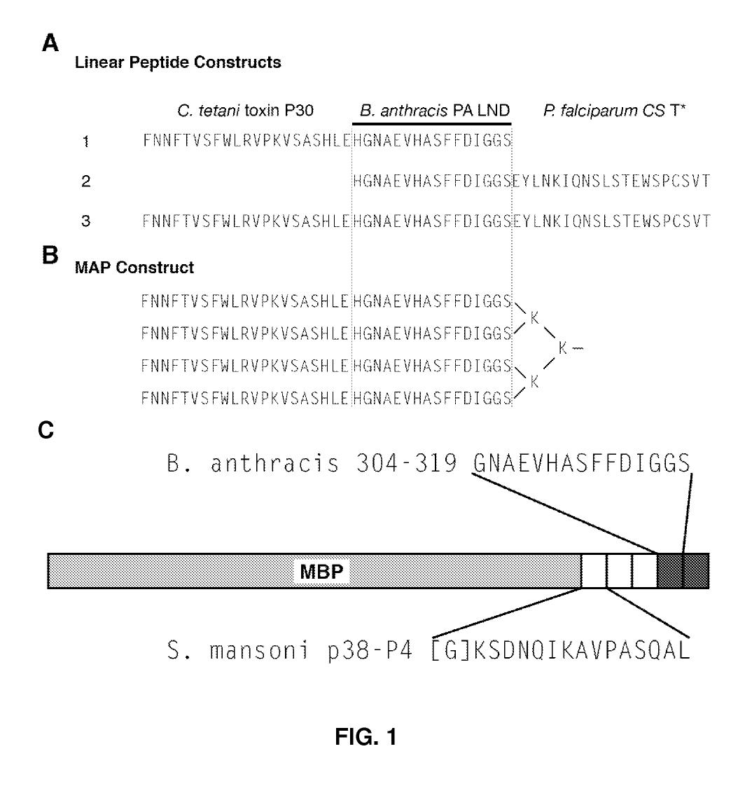

FIG. 2 shows a wire frame model of the PA monomer derived from the 1TZN structure, oriented to display the LND in profile with selected residues labeled using the single letter amino acid code followed by the sequence number. The receptor-binding domain is at the C-terminus and the LF and EF binding region is near the N-terminus.

FIG. 3 shows a space-filling model of the PA monomer derived from the 1TZN crystal structure, with the loop neutralizing determinant (LND) identified.

FIG. 4 shows lymph node proliferative responses to the PA 305-319 and P30 peptides in 3 strains of mice. Lymph node proliferative responses to the PA 305-319 peptide or to the P30 peptide in C57BL/6 (A), SJL (B) or BALB/c (C) mice. 5 mice/group were immunized s.c. at the base of the tail with 12 nanomoles of the PA 305-319 peptide or the P30 peptide in an emulsion with CFA. Ten days later, periaortic and inguinal lymph nodes were restimulated with the indicated concentrations of test antigen for 72 hours and proliferation was assessed by scintillation counting following incubation with tritiated thymidine. Stimulation indices represent proliferation in the presence of test antigen/proliferation in the media alone. Error bars=SEMs

FIG. 5 shows antibody and TNA titers in C57BL/6 and SJL mice following immunization with PA, P30-PA305-319 peptide or the P30 peptide alone. Five mice per group of C57BL/6 (A,C) and SJL mice (B,D) were immunized 4 times at two-week intervals with either 50 μg of PA, or 12 nanomoles of either the P30-PA305-319 peptide or the P30 peptide mixed with 10 μg of Quil A adjuvant. Ten days after the last immunization, mice were bled and serum was analyzed by ELISA for responses to immobilized PA (A,B) or in the in vitro toxin neutralization assay (C,D) performed as described in Materials and Methods. The lower limit of detection for both assays is 16, and data below this level is indicated with a triangle. Bar charts represent geometric means. Error bars=SEMs

FIG. 6 shows the results of Panel (A) ELISA and Panel (B) TNA assays on individual rabbit antiserum following 4 immunizations with the P30-PA305-319 peptide using CFA/IFA adjuvant. TNA titers were determined as described in the example and the results are expressed as the percentage neutralization compared to a hyperimmune rabbit serum raised against soluble PA.

FIG. 7 shows robust T cell proliferation to the MBP carrier protein in all strains of mice tested, as shown in FIG. 7 Panels (B)-(E), except as shown in Panel (A), C3H mice. Results shown reflect in vitro T cell proliferative responses from pooled lymph node cells removed from four inbred strains of mice and an F1 following immunization with MBP. Four mice per group were immunized subcutaneous (s.c.) at the base of the tail with 50 μg of MBP in a 1:1 emulsion with CFA. Seven days later, periaortic and inguinal lymph node cells were restimulated in vitro with MBP (circles) or with two irrelevant peptides (squares) and (triangles) at the indicated concentrations. The highest line in all figures demonstrates responses to the MBP test antigen. Stimulation index=CPM in the presence of test antigen/CPM in the presence of media only. Error bars=SEMs.

FIG. 8 shows in vitro T cell proliferative responses from pooled lymph node cells removed from four inbred strains of mice following immunization with the SM peptide. Four mice per group were immunized s.c. at the base of the tail with 50 μg of the SM peptide in a 1:1 emulsion with CFA. Seven days later, periaortic and inguinal lymph node cells from C3H (circles), BALB/c (triangles), SJL (diamonds) or C57BL/6 (squares) mice were restimulated in vitro with SM peptide (circles) at the indicated concentrations. Stimulation index=CPM in the presence of test antigen/CPM in the presence of media only.

FIG. 9 shows a bar chart depicting geometric mean antibody titers to immobilized PA in serum from mice immunized 4 times with the SM(3)PA305(2) immunogen in Alum/MPL adjuvant. Mice were bled ten days after their last booster immunization. There was no PA-specific immunity in any mouse pre-bleed serum (not shown). Titers were computed for each serum by using nonlinear regression to fit a variable slope sigmoidal equation to the serial dilution data set using Prism 5. Error bars=SEMs.

FIG. 10 shows ELISA titers in Panel (A) and PA LeTx neutralization titers in Panel (B) from 4 rabbits immunized at two week intervals with the SM(3)PA305(2) immunogen in an emulsion with CFA for priming immunizations and with IFA for all booster immunizations. Serum was obtained 10 days after the booster immunizations. For antibody titers, serum underwent serial two-fold dilutions and the titer was considered the reciprocal dilution at one-half the maximal signal. LeTx neutralization EC₅₀ titers were performed as described in the example and is expressed as a percentage of the control anti-PA neutralization.

FIG. 11 shows ELISA results in the form of bar charts from two rabbits immunized with the SM(3)PA305(2) immunogen. Rabbits 652 and 653 were immunized at two-week intervals with the SM(3)PA305(2) immunogen with an Alhydrogel/MPL adjuvant. Rabbits were bled 10 days after each immunization and serum was analyzed by ELISA Panel (A) and with the TNA, Panel (B). The assay results were repeated 4 times.

FIG. 12 shows antibody and toxin neutralizing responses in the serum of rabbits immunized with the MAP peptide or with full length PA. Shown are antibody responses to immobilized 305-319 peptide sequence (A) or to PA (B) and TNA activity (C) from the serum of rabbits immunized 5 times at 2 week intervals with either the MAP peptide (MR1-MR3) or PA (PR1, PR2) as described under material and methods and as depicted graphically in the time line. Serum responses are from test bleeds obtained 10 days after the 5^{th} immunization. For analysis of peptide reactivity, the immobilized antigen was a recombinant protein displaying 2 copies of the peptide sequence, a.a. 299-327 from PA. Antibody and TNA titers were determined as described under the materials and methods section and are expressed as the reciprocal of the EC50 or ED50, respectively. The lower limit of assay detection for the ELISA and TNA is 16, and sample data below this level are indicated with an asterik (*). Error bars represent SEMs.

FIG. 13 shows antibody responses in the sera of rabbits immunized with the MAP peptide. Sera obtained from MAP peptide-immune rabbits 10 days after the indicated immunizations were tested by ELISA for reactivity with immobilized peptide sequence (A) or with PA (B). For analysis of peptide reactivity, the immobilized antigen was a recombinant protein displaying 2 copies of the peptide sequence, a.a. 299-327 from PA. Antibody titers were determined as described under materials and methods and are expressed as the reciprocal of the EC50. As controls, immunoreactivity with immobilized PA from the antisera of the two PA-immune rabbits is shown in 2B (diamonds) with the horizontal line representing GMT. Responses from the control rabbits are from antisera obtained at approximately week 10.

FIG. 14 shows an analysis of the MAP-peptide antisera in the TNA. Sera obtained from the second group of MAP peptide-immune rabbits 10 days after the indicated immunizations were tested in the TNA as described under materials and methods. The left y-axis corresponds to the reciprocal of the ED50 neutralization titers and the right y-axis denotes the ED50 titers normalized to the geometric mean ED50 neutralization titers from the sera of the two PA-immune control rabbits obtained 10 days after their 5^{th} immunization (diamonds). There was no detectable neutralization in the pre-immune sera from any of the rabbits.

FIG. 15 shows that MAP-immune antisera contains highly avid antibody. Shown are the avidity maturation data from the second cohort of MAP-immune rabbits (MR4-MR6) and individual avidity data from the MAP-immune rabbit, MR3, and the two positive control PA-immune rabbits (PR1, PR2) as determined through use of a chaotrope ELISA as described under materials and methods. The avidity index was determined from sera obtained 10 days after the indicated immunizations from the second group of MAP peptide-immune rabbits, or after the 5^{th} immunization for rabbits MR3, PR1 and PR2.

FIG. 16 shows an effect of pre-incubation with the 304-319 peptide or an irrelevant peptide on the neutralization titers in the MAP and PA antisera. Antisera from the three MAP-immune rabbits obtained after the 6^{th} immunization and from the PA-immune rabbits after the 5^{th} immunization were incubated with 20 μM of the indicated peptides for 30 minutes prior to assessment in the TNA. TNA titers are expressed as the reciprocal of the ED50 and were determined as described under material and methods. The lower limit of assay detection for the TNA is 16; samples below this limit are indicated with an asterik (*).

FIG. 17 shows comparative immunogenicity of P30 containing T-B peptides targeting the LND of PA. Groups of five B6C3F1 mice (C57BL/6×C3H) were immunized s.c. four times at two week intervals with 12 nanomoles of a synthetic peptide comprised of the P30 helper T cell epitope colinearly synthesized to an LND region peptide as shown above. Serum was collected 10 days after the final immunization and analyzed by ELISA for reactivity with immobilized PA and in the TNA. Bar charts=(geometric mean TNA ED₅₀ titer/geometric mean EC₅₀ antibody titer)×100.

FIG. 18 shows responses of rabbits immunized with the MAP304 and MAP305 peptides. Antibody responses to immobilized peptide comprising a.a. 299-327 from PA (A and B) to immobilized PA (C and D) and TNA activity (E and F) in sera from rabbits immunized at two-week intervals with either the MAP304 or the MAP305 peptides. Serum for analysis was obtained 10 days after the indicated bleed. In E and F, the left y-axis corresponds to the EC₅₀ neutralization titer and the right y-axis refers to the (ED₅₀ of the experimental serum)/(the mean EC₅₀ neutralization titer of the positive control anti-PA serum)×100. The triangles represent the antibody and neutralization titers from the two rabbits comprising the PA control antisera which were immunized as described. There was no detectable neutralization in the pre-immune serum from any of the rabbits (not shown). Each circle or square represents an individual rabbit data point and the horizontal lines are geometric means. The immunization protocol is diagrammed at the bottom of the figure.

FIG. 19 shows a comparison of antibody and TNA titers among rabbits immunized with LND MAPs containing the P30 helper epitope or no helper epitope. Comparison of geometric mean antibody titer (A) or neutralization (B) in sera from rabbits immunized 6 times with either the MAP304 or MAP305, both of which contain the P30 helper epitope, or a MAP displaying the 305-319 without a helper epitope. For comparison of neutralization, all titers are normalized to the neutralization from control PA antisera. Error bars=SEMs.

FIG. 20 shows durability of neutralizing antibody responses in MAP-304-immune rabbits. Serial analysis of the antibody (A) and TNA (B) titers from the three MAP304 rabbits at the time points indicated following their sixth and final immunization. Diamonds represent the antibody and TNA titers from the two rabbits which comprise the PA control antiserum.

FIG. 21 shows an effect of pre-incubation of MAP-304 and PA83 antiserum with peptides on in vitro toxin neutralization assay. Antiserum from the three MAP304-immune rabbits procured after the 4^{th} immunization, and control PA antisera from two rabbits, were incubated with 20 μM of the indicated peptides for 30 minutes prior to assessment in the TNA. The lower limit of detection for the TNA assay is 16, and data below this level is indicated with a triangle.

FIG. 22 shows determination and comparison of LND peptide-specific antibody affinities in the sera from rabbits immunized with either LND MAPs containing the P30 helper T cell epitope or an LND MAP without a helper T cell epitope. LND-peptide specific IC₅₀s were determined for antisera of rabbits immunized with the MAP304 or MAP305 (A) and for antisera from rabbits immunized with a MAP peptide without a helper epitope (B) from a previous study. Antisera from two rabbits immunized with the MAP peptide without a helper epitope which had no significant antibody responses were not considered in this analysis. Peptide IC₅₀s were determined as described. Panel (C) shows mean peptide-specific IC₅₀s in antisera from rabbits immunized with the LND MAPs containing the P30 helper epitope were significantly lower than the mean IC₅₀s from the antisera of rabbits immunized with the MAP without a helper epitope (*, p=0.019). Error bars=SEMs.

FIG. 23 shows correlation of peptide avidity and antibody titer with neutralization titers among MAP-immune rabbits. A significant negative correlation between peptide IC₅₀s and normalized TNA titers (A) and a significant positive correlation between antibody titers and TNA titers (B) was observed among all rabbits immunized with LND-containing MAP peptides. TNA titers from all MAP-immune rabbits were normalized to the control PA antiserum as described in Materials and Methods. Correlations were determined using Spearman's rho correlation. Note two data points overlap along the x-axis at an antibody titer of approximately zero in panel (B).

FIG. 24 shows immunoreactivity with PA and with the 305-319 peptide sequence from sera of rabbits immunized with PA. Seven rabbits received priming immunizations with PA in CFA and then were boosted 2 weeks later with PA in IFA (A), and four rabbits received priming immunizations with PA in Alhydrogel and were boosted twice at two week intervals (B). Ten days after the final booster immunization (day 24 for rabbits in A, day 34 for rabbits in B), rabbits were bled and individual rabbit serum (circles) was analyzed for immunoreactivity with either PA, the 305-319 peptide sequence, or an irrelevant peptide sequence. For analysis of peptide reactivity, a recombinant protein displaying two copies per molecule of a.a. 305-319 from PA, or an irrelevant peptide sequence, was immobilized on the ELISA plates as described under material and methods. As a positive control, the immunoreactivity of the serum from four MAP peptide-immune rabbits (triangles) is also shown in 6A. There was no significant difference in GMTs from the sera of the rabbits immunized with PA in Freund's (A) when tested on either immobilized 305-319 peptide sequence or the irrelevant peptide sequence (Student's t-test, p=0.8513). The lower limit of assay detection for the ELISA is 16; samples below this limit are plotted at 16. Horizontal lines represent GMTs.

FIG. 25 shows immunoreactivity with PA and with the 305-319 peptide sequence from serum of rabbits inoculated one time with recombinant adeno-associated vectors expressing PA63. Twelve rabbits received a single inoculation with rAAV vectors expressing PA63. Of these 12 rabbits, 6 also received rAAV expressing LF. Eight weeks after being inoculated, all rabbits were bled and sera was analyzed by ELISA for immunoreactivity with PA, with the 305-319 peptide sequence, or with an irrelevant peptide sequence. For analysis of peptide reactivity, a recombinant protein displaying two copies per molecule of a.a. 305-319 from PA, or an irrelevant peptide sequence, was immobilized on the ELISA plates as described under materials and methods. The lower limit of assay detection for the ELISA is 16; sample data below this limit are plotted at 16. Horizontal lines represent GMTs.

FIG. 26 in panel (A) shows sera reacted with PA by ELISA with EC50 titers in the range of 600-1000; a high titer rabbit anti-LND sera is shown as a control. Panel (B) shows that the human and macaque sera samples had no detectable antibody against the LND, while the rabbit anti-LND sera react strongly with the 299-327 sequence.

FIG. 27 shows a survival plot of rabbits challenged with anthrax Ames Strain spore inhalation challenge in rabbits immunized with various immunogens rPA83. rMBP-SM3-(PA305-319)₂, PA304-T*MAP, P30-PA304MAP & T*-PA304MAP, P30-PA304MAP & PA304-T*MAP and T*PA304MAP.

### DETAILED DESCRIPTION

Example embodiments will now be described more fully with reference to the accompanying drawings.

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom.

The following definitions and non-limiting guidelines must be considered in reviewing the description of the technology set forth herein.

The headings (such as “Introduction” and “Summary”) and sub-headings used herein are intended only for general organization of topics within the present technology, and are not intended to limit the disclosure of the present technology or any aspect thereof. In particular, subject matter disclosed in the “Introduction” may include novel technology and may not constitute a recitation of prior art. Subject matter disclosed in the “Summary” is not an exhaustive or complete disclosure of the entire scope of the technology or any embodiments thereof. Classification or discussion of a material within a section of this specification as having a particular utility is made for convenience, and no inference should be drawn that the material must necessarily or solely function in accordance with its classification herein when it is used in any given composition.

The citation of references herein does not constitute an admission that those references are prior art or have any relevance to the patentability of the technology disclosed herein. Any discussion of the content of references cited in the Introduction is intended merely to provide a general summary of assertions made by the authors of the references, and does not constitute an admission as to the accuracy of the content of such references. All references cited in the “Detailed Description” section of this specification are hereby incorporated by reference in their entirety.

The description and specific examples, while indicating embodiments of the technology, are intended for purposes of illustration only and are not intended to limit the scope of the technology. Moreover, recitation of multiple embodiments having stated features is not intended to exclude other embodiments having additional features, or other embodiments incorporating different combinations of the stated features. Specific examples are provided for illustrative purposes of how to make and use the compositions and methods of this technology.

As used herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the technology.

As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features.

Although the open-ended term “comprising,” as a synonym of terms such as including, containing, or having, is used herein to describe and claim the present invention, the invention, or embodiments thereof, may alternatively be described using more limiting terms such as “consisting of” or “consisting essentially of” the recited ingredients. As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

“A” and “an” as used herein indicate “at least one” of the item is present; a plurality of such items may be present, when possible. “About” when applied to values indicates that the calculation or the measurement allows some slight imprecision in the value (with some approach to exactness in the value; approximately or reasonably close to the value; nearly). If, for some reason, the imprecision provided by “about” is not otherwise understood in the art with this ordinary meaning, then “about” as used herein indicates at least variations that may arise from ordinary methods of measuring or using such parameters. In addition, disclosure of ranges includes disclosure of all distinct values and further divided ranges within the entire range.

The present disclosure provides novel compositions and methods for producing an immune response in a mammalian subject, preferably a human subject that mitigates or prevents the effects of anthrax toxin and thereby helps to protects cells of immune individuals from the morbidity and mortality that would otherwise result from anthrax infection.

Analysis of the structure and function of anthrax protective antigen reveals a loop referred to as the 2β2-2β3 loop which is poorly ordered in monomeric PA but which, in PA homo-heptameric pores, associates with like structures in neighboring molecules to form the transmembrane channel through which lethal factor and edema factor translocate to the cytosol for ultimate intoxication of the cell through their enzymatic activities. The present disclosure provides immunogens that elicit antibodies that bind to an epitope in the 2β2-2β3 loop, and thereby mediate neutralization of toxin function. As used herein, the term “immunogen” and “immunostimulatory polypeptide” are synonymous and include polypeptides that are capable of eliciting a B cell response and/or a T cell response in a subject treated with or vaccinated with an immunogen of the present disclosure, including the polypeptides set forth in SEQ ID NOs: 1-6 and their functional variants and immunostimulatory fragments thereof.

The present disclosure provides synthetic peptides and recombinant protein immunogens that elicit neutralizing antibody responses against this Loop Neutralizing Determinant (LND), and further disclose variations that incorporate helper T cell epitopes so as to augment the antibody response against the LND. These compositions are useful as the active ingredients of vaccines for prevention of inhalation anthrax.

Accordingly, the present disclosure provides immuno stimulatory compositions based on the amino acid sequence of the 2β2-2β3 loop of Bacillus anthracis PA. Immunostimulatory means the ability to stimulate immune responses that result in antibodies that specifically bind within this loop in such a manner that they interfere with toxin function to an extent that at least partially or fully abrogates or neutralizes its action on cells. The structure recognized by such antibodies defines the loop neutralizing determinant (LND) of PA.

In some embodiments, the vaccine is a polypeptide containing the Bacillus anthracis PA residues 308-318 of SEQ ID NO: 1 and/or a functional variant thereof. This polypeptide segment represents a minimal representation of the LND and consequently will typically be incorporated in a larger peptide or protein sequence that may provide additional immunologic or practical advantages.

Thus in some embodiments, contiguous N- or C-terminal residues are included, which are derived from Bacillus anthracis protective antigen 299-327 of SEQ ID NO: 2. In some embodiments, the sequence of SEQ ID NO: 3 is included as a highly functional and practical representation of the LND.

The LND sequences may be modified as long as they retain their immunostimulatory effect, which can be measured by their ability to elicit anthrax lethal toxin-neutralizing antibody responses. As such, in some embodiments, the disclosure encompasses functional variants of the peptides of the disclosure. As used herein, a “functional variant” or “variant” of a peptide is a peptide which contains one or more modifications to the primary amino acid sequence of the peptides of the present disclosure while retaining the immunostimulatory effect disclosed herein. Functional variants can be readily determined using antibody binding assays, such as an ELISA, as is routine in the art. For example, in some embodiments, functional variants should retain at least 50% of the activity of the original peptide. In some embodiments, the functional variant should retain at least 60%, 70%, 80%, or 90% of the activity of the original peptide. Quantitative binding affinity may be used to determine the activity of the functional variant and to select a functional variant that meets one of the aforementioned activity thresholds.

In the context of the present disclosure, a homologous sequence is taken to include an amino acid sequence which is at least 60%, 70%, 80% or 90% identical, preferably at least 95% or 98% identical at the amino acid level with a particular sequence. In particular, homology should typically be considered with respect to those regions of the sequence known to be essential for antigenicity rather than non-essential neighboring sequences. Regions that are conserved across family members can have relatively high homology scores which assist in defining functional molecules. Although homology can also be considered in terms of similarity (i.e., amino acid residues having similar chemical properties/functions), in the context of the present disclosure it is preferred to express homology in terms of sequence identity.

Homology comparisons can be conducted by eye, or more usually, with the aid of readily available sequence comparison programs. These commercially available computer programs can calculate % homology between two or more sequences. Particular sequences of the disclosure may therefore be modified for use in the present disclosure. Typically, modifications are made that maintain the antigenicity of the sequence. Thus, in one embodiment, amino acid substitutions may be made, for example from 1, 2, or 3 to 10, 20 or 30 substitutions, provided that the modified sequence retains at least about 25% to 50% of, or substantially the same activity. However, in alternative embodiments, modifications to the amino acid sequences of a polypeptide of the disclosure may be made intentionally to reduce the biological activity of the polypeptide.

For example, truncated polypeptides that remain capable of binding to target molecule but lack functional effector domains may be useful as inhibitors of the biological activity of the full length molecule. In general, preferably less than 20%, 10%, or 5% of the amino acid residues of a variant or derivative are altered as compared with the corresponding region depicted in the sequence listings.

The terms “polypeptide,” “protein,” and “peptide” include compositions of the present disclosure that also include “analogs,” or “conservative variants” and “mimetics” (“peptidomimetics”) with structures and activity that substantially correspond to the exemplary sequences. Thus, the terms “conservative variant” or “analog” or “mimetic” also refer to a polypeptide or peptide which has a modified amino acid sequence, such that the one or more changes do not substantially alter the polypeptide's (the conservative variant's) structure and/or activity (e.g., immunogenicity, ability to bind to human antibodies, etc.), as defined herein. These include conservatively modified variations of an amino acid sequence, i.e., amino acid substitutions, additions or deletions of those residues that are not critical for protein activity, or substitution of amino acids with residues having similar properties (e.g., acidic, basic, positively or negatively charged, polar or non-polar, etc.) such that the substitutions of even critical amino acids does not substantially alter structure and/or activity.

Conservative substitution tables providing functionally similar amino acids are well known in the art. For example, one exemplary guideline to select conservative substitutions includes (original residue followed by exemplary substitution): ala/gly or ser; arg/lys; asn/gln or his; asp/glu; cys/ser; gln/asn; gly/asp; gly/ala or pro; his/asn or gln; ile/leu or val; leu/ile or val; lys/arg or gln or glu; met/leu or tyr or ile; phe/met or leu or tyr; ser/thr; thr/ser; trp/tyr; tyr/trp or phe; val/ile or leu. An alternative exemplary guideline uses the following six groups, each containing amino acids that are conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K); 5) Isoleucine (1), Leucine (L), Methionine (M), Valine (V); and 6) Phenylalanine (F), Tyrosine (Y), Tryptophan (W); (see also, e.g., Creighton (1984) Proteins, W. H. Freeman and Company; Schulz and Schimer (1979) Principles of Protein Structure, Springer-Verlag). One of skill in the art will appreciate that the above-identified substitutions are not the only possible conservative substitutions. For example, for some purposes, one may regard all charged amino acids as conservative substitutions for each other whether they are positive or negative. In addition, individual substitutions, deletions or additions that alter, add or delete a single amino acid or a small percentage of amino acids in a sequence can also be considered “conservatively modified variations.”

Conservative substitutions may be made, for example according to the Table 1 below. Amino acids in the same block in the second column and preferably in the same line in the third column may be substituted for each other:

TABLE 1
CONSERVATIVE AMINO ACID REPLACEMENTS

| For Amino Acid | Code | Replace with any of |
|---|---|---|
| Alanine | A | D-Ala, Gly, beta-Ala, L-Cys, |
| | | D-Cys |
| Arginine | R | D-Arg, Lys, D-Lys, homo-Arg, |
| | | D-homo-Arg, Met, Ile, D-Met, |
| | | D-Ile, Orn, D-Orn |
| Asparagine | N | D-Asn, Asp, D-Asp, Glu, |
| | | D-Glu, Gln, D-Gln |
| Aspartic Acid | D | D-Asp, D-Asn, Asn, Glu, |
| | | D-Glu, Gln, D-Gln |
| Cysteine | C | D-Cys, S-Me-Cys, Met, D-Met, |
| | | Thr, D-Thr |
| Glutamine | Q | D-Gln, Asn, D-Asn, Glu, |
| | | D-Glu, Asp, D-Asp |
| Glutamic Acid | E | D-Glu, D-Asp, Asp, Asn, |
| | | D-Asn, Gln, D-Gln |
| Glycine | G | Ala, D-Ala, Pro, D-Pro, |
| | | .beta.-Ala, Acp |
| Isoleucine | I | D-Ile, Val, D-Val, Leu, |
| | | D-Leu, Met, D-Met |
| Leucine | L | D-Leu, Val, D-Val, Leu, |
| | | D-Leu, Met, D-Met |
| Lysine | K | D-Lys, Arg, D-Arg, homo- |
| | | Arg, D-homo-Arg, Met, D-Met, |
| | | Ile, D-Ile, Orn, D-Orn |
| Methionine | M | D-Met, S-Me-Cys, Ile, |
| | | D-Ile, Leu, D-Leu, Val, |
| | | D-Val |
| Penylalanine | F | D-Phe, Tyr, D-Thr, L-Dopa, |
| | | His, D-His, Trp, D-Trp, |
| | | Trans-3,4, or 5-phenyl- |
| | | proline, cis-3,4, or |
| | | 5-phenylproline |
| Proline | P | D-Pro, L-I-thioazolidine-4- |
| | | carboxylic acid, D-or L-1- |
| | | oxazolidine-4-carboxylic acid |
| Serine | S | D-Ser, Thr, D-Thr, allo-Thr, |
| | | Met, D-Met, Met(O), D-Met(O), |
| | | L-Cys, D-Cys |
| Threonine | T | D-Thr, Ser, D-Ser, allo-Thr, |
| | | Met, D-Met, Met(O), D-Met(O), |
| | | Val, D-Val |
| Tyrosine | Y | D-Tyr, Phe, D-Phe, L-Dopa, |
| | | His, D-His |
| Valine | V | D-Val, Leu, D-Leu, Ile, D-Ile, |
| | | Met, D-Met |

Amino acid substitutions may include the use of non-naturally occurring analogs, for example to increase blood plasma half-life of a therapeutically administered polypeptide.

The terms “mimetic” and “peptidomimetic” refer to a synthetic chemical compound that has substantially the same structural and/or functional characteristics of the polypeptides of the present disclosure (e.g., ability to bind, or “capture,” human antibodies in an ELISA). The mimetic can be either entirely composed of synthetic, non-natural analogues of amino acids, or, is a chimeric molecule of partly natural peptide amino acids and partly non-natural analogs of amino acids. Mimetics can also incorporate any amount of natural amino acid conservative substitutions as long as such substitutions also do not substantially alter the mimetics' structure and/or activity. As with polypeptides of the present disclosure which are conservative variants, routine experimentation will determine whether a mimetic is within the scope of the disclosure, i.e., that its structure and/or function is not substantially altered.

Polypeptide mimetic compositions can contain any combination of non-natural structural components, which are typically from three structural groups: a) residue linkage groups other than the natural amide bond (“peptide bond”) linkages; b) non-natural residues in place of naturally occurring amino acid residues; or c) residues which induce secondary structural mimicry, i.e., to induce or stabilize a secondary structure, e.g., a beta turn, gamma turn, beta sheet, alpha helix conformation, and the like. A polypeptide can be characterized as a mimetic when all or some of its residues are joined by chemical means other than natural peptide bonds. Individual peptidomimetic residues can be joined by peptide bonds, other chemical bonds or coupling means, such as, e.g., glutaraldehyde, N-hydroxysuccinimide esters, bifunctional maleimides, N,N′-dicyclohexylcarbodiimide (DCC) or N,N′-diisopropylcarbodiimide (DIC). Linking groups that can be an alternative to the traditional amide bond (“peptide bond”) linkages include, e.g., ketomethylene (e.g., —C═O)—CH₂— for —C(═O)—NH—), aminomethylene (CH₂—NH), ethylene, olefin (CH═CH), ether (CH₂—O), thioether (CH₂—S), tetrazole (CN₄—), thiazole, retroamide, thioamide, or ester (see, e.g., Spatola (1983) in Chemistry and Biochemistry of Amino Acids, Peptides and Proteins, Vol. 7, pp 267-357, “Peptide Backbone Modifications,” Marcell Dekker, N.Y.). A polypeptide can also be characterized as a mimetic by containing all or some non-natural residues in place of naturally occurring amino acid residues; non-natural residues are well described in the scientific and patent literature.

Modifications, which generate functional variants of the peptides of the present disclosure, may also comprise the addition of amino acids at either end of the peptides, i.e., at the N or C termini. Again, any well-known method for preparing modified or variant peptides can be employed, such as synthesis of the modified or variant peptide or its recombinant production using a mutated nucleic acid molecule. The peptides of the present disclosure may also be modified to be more resistant to hydrolysis by proteases, such as by containing D-amino acids or one or more non-hydrolyzable peptide bonds linking amino acids. Non-hydrolyzable peptide bonds are well-known in the art and may include -psi[CH₂NH]-reduced amide peptide bonds, -psi[COCH₂]-ketomethylene peptide bonds, -psi[CH(CN)NH]-(cyanomethylene)amino peptide bonds, -psi[CH₂CH(OH)]-hydroxyethylene peptide bonds, -psi[CH₂O] peptide bonds, and -psi[CH₂S]-thiomethylene peptide bonds. The peptides of the present disclosure may also comprise unnatural and unusual amino acids and amino acid analogs, such as ornithine, norleucine, L-malonyltyrosine and others known to those of skill in the art. Alternatively, the peptides of the present disclosure and their functional variants may be rendered more resistant to degradation or their structural stability may be increased by the inclusion of nonpeptide moities. Preferably, the nonpeptide moieties permit the peptides to retain their natural conformation, or stabilize an optimized bioactive confirmation. Examples of suitable substitutions include D-isomer amino acids, N-methyl amino acids, L-isomer amino acids, modified L-isomer amino acids and cyclized derivatives. Such peptide mimetics can be tested in molecular or cell-based binding assays as described herein to assess the effect of the substitution(s) on conformation and/or activity. Procedures of medicinal chemistry may be applied by one skilled in the art using routine experimental methods of e.g. rational drug design, molecular modeling based on structural information from nuclear magnetic resonance or X-ray diffraction data, and other computational methods. Thus, the disclosure includes all of the foregoing modifications to the peptides.

Other suitable substitutions are easily established by the person of skill and may additionally be determined by reference to publications such as Voet, Biochemistry, Wiley, 1990; Stryer Biochemistry 4th Ed., Freeman N.Y., 1995; Peptide Chemistry. A Practical Textbook, 2nd ed., Miklos Bodanszky, Springer-Verlag, Berlin, 1993; Principles of Peptide Synthesis, 2nd ed., Miklos Bodanszky, Springer-Verlag, Berlin, 1993; Chemical Approaches to the Synthesis of Peptides and Proteins, P. Lloyd-Williams, F. Albericio, E. Giralt, CRC Press, Boca Raton, 1997; Bioorganic Chemistry: Peptides and Proteins, S. M. Hecht, Ed., Oxford Press, Oxford, 1998, Synthetic Peptides: A User's Guide, Gregory A. Grant (Editor), Oxford University Press, 2002, and the like, all of which are incorporated by reference herein.

Functional variants can be identified by preparing a candidate polypeptide containing the variant, combining it with an appropriate immunologic adjuvant such as Freund's complete and incomplete adjuvant, using this formulation to immunize an appropriate animal such as rabbits, and after two or more immunizations, obtaining sera from the rabbits and testing its ability to antibodies neutralize anthrax lethal toxin in a standard assay.

Immunostimulatory B. anthracis immunogen polypeptides of the present disclosure also include fragments of full length polypeptides and variants thereof. Exemplary fragments include those, which include an epitope that is recognized by an anti-PA, anti-LF and anti-EF antibody. Suitable fragments will be at least above 5, e.g., 10, 12, 15 or 20 amino acids in length. They may also be less than 200, 100 or 50 amino acids in length. Polypeptide fragments of the proteins and allelic and species variants thereof may contain one or more (e.g., 2, 3, 5, 10 or 20) substitutions, deletions or insertions, including conserved substitutions. Where substitutions, deletion and/or insertions have been made, for example by means of recombinant technology, preferably less than 20%, 10% or 5% of the amino acid residues depicted in the sequence listings are altered. Particularly preferred fragments include those having antigenic domains.

Peptide segments that represent the LND are essential as B cell epitopes for eliciting the required antibody specificity, but may be deficient in, or devoid of, the ability to stimulate helper T cell immunity required for those antibody responses. Therefore, in some embodiments, pharmaceutical and vaccine compositions of the present disclosure contain linked polypeptide sequences, helper T cell epitopes, that stimulate helper T lymphocyte responses. Such vaccines can include helper T lymphocyte epitopes selected from the group consisting of Clostridium tetani toxin 947-967, Clostridium tetani toxin 830-844, Plasmodium falciparum circumsporozoite protein 326-345, Shistosoma mansoni 38 kDa Soluble Egg Antigen 235-249, measles virus fusion protein 288-303, among others known to those skilled in the art.

The peptides and polypeptides of the present disclosure can be produced by chemical synthesis or may be recombinant in origin. In some embodiments, the vaccine can be a linear synthetic peptide which may have one or more helper T cell epitopes colinearly synthesized N- or C-terminal to the B cell epitope representing the LND, as in the compositions of SEQ ID NO: 4 and SEQ ID NO: 5 respectively, or may have helper epitopes at both termini, as in the case of SEQ ID NO: 6. In some embodiments, the B cell epitope segment in the peptide or protein is cyclized.

In some embodiments, the polypeptides can comprise amino acid residues located at or near the N and/or C termini of the peptides having side chains suitable for the formation of an intramolecular crosslink for purposes of cyclizing the peptides. Suitable residues for crosslinking are well known to a person of skill in the art and may comprise disulfide (cysteine-cysteine), thioether (cysteine-electrophile, such as bromoacetyl, maleimidyl etc.) and other bonds. In some embodiments, the crosslink for cyclization is provided by a disulfide bridge between cysteine residues located at or near the N and/or C termini of the peptides. In some embodiments, terminal spacer residues are added to the peptides of the disclosure in order to ensure the spatial accessibility of the crosslinking residues, such as for the incorporation of the cyclic peptide into liposomes, virosomes, or other suitable delivery vehicles. For example, a GGC sequence may be added to the N-terminus and an additional glycine residue at the C-terminus, but many other spacer sequences known to those of skill in the art may be used for the purposes of the present disclosure, as long as the side chains of the spacer residues are small enough so as not to sterically interfere with the intramolecular crosslink. Examples of suitable spacer residues comprise amino acids such as alanine, serine, asparagine, glutamine, or glycine. Thus, in some embodiments of the disclosure, LND peptides are cyclized by the formation of an intramolecular crosslink. The cyclization of the peptides of the disclosure provides for the emulation of the native three-dimensional structure of the LND and is intended to further optimize neutralizing antibody responses against the PA. Again, internal crosslinks can be introduced via a number of residues, both natural and synthetic, which are well known in the art. In some embodiments, terminally positioned (located at or near the N- and/or C-termini of the peptide) cysteine residues can be used to cyclize the peptides through a disulfide bond.

In some embodiments, the peptides of the present disclosure and functional variants thereof are cyclized by the use of a template. One advantage of using such widely available templates is their rigidity that may stabilize the three-dimensional conformation of the cyclized peptides more effectively than the use of internal crosslinks which typically introduce several rotatable bonds, thereby destabilizing the cyclized peptide structure. Suitable templates for the cyclization of peptide chains are well known in the art and may be tricyclic (Beeli et al., Helvetica Chimica Acta 79: 2235 2248, 1996), diketopiperazine-based (Bisang et al., Helvetica Chimica Acta 79: 1825 1842, 1996), bicyclic, such as a template derived from differentially substituted diaminoprolines (Pfeifer et al., Chem. Commun. 1977 78, 1998) or heterochiral diprolines (Favre et al. J. Am. Chem. Soc. 121: 2679 2685, 1999), to name only a few.

In some embodiments, the peptides of the present disclosure and functional variants thereof are synthesized in linear form and purified prior to conjugation to a branching or dendrimeric core structure.

In some embodiments, the polypeptide segments are configured in a branching manner by synthesis from, or conjugation to, a suitable core or backbone structure. In some embodiments, polypeptides are synthesized from four or eight initiation sites comprised of the α- and ε-amines of a branching lysine core so as to result in what has been termed a multiple antigen structure.

In some embodiments, the polypeptide segment containing SEQ ID NO: 1 is repeated. In recombinant polypeptides, one or more B cell epitopes representing the LND can be colinear synthesized with repeated T cell epitopes or a fusion protein. In some embodiments, the fusion protein can contain an Escherichia coli maltose binding protein or derivatives thereof.

In some embodiments, the polypeptide can be encoded by DNA and expressed in the vaccinated individual. This may be accomplished using any one of many recombinant vaccine expression vectors, including viral, bacterial, and “naked” DNA approaches well known to those of ordinary skill in the art.

Initial doses of vaccine can typically be followed by booster doses, following immunization protocols standard in the art, and their effect may be potentiated by adjuvants or cytokines well known to those skilled in the art. Again, the peptides of the present disclosure, as well as functional variants thereof, may be encapsulated by or attached to the surface of the delivery vehicles in linear or cyclized form. The present disclosure also provides for the administration of the immunostimulatory LND-targeted peptides in a suitable pharmaceutical formulation. Administration or administering is meant as providing one or more peptides or peptide-containing compositions of the disclosure to an individual in need of treatment or prevention of anthrax. Such a composition which contains one or more of the peptides and/or peptide containing compositions of the present disclosure, including functional variants thereof, as the principal or member active ingredient, for use in the treatment or prevention of anthrax, can be administered in a wide variety of therapeutic dosage forms in the conventional vehicles for topical, oral, systemic, local, and parenteral administration.

The present disclosure therefore provides pharmaceutical and vaccine compositions for parenteral administration which comprise a solution of the peptides and their functional variants dissolved or suspended in an acceptable pharmaceutical excipient or carrier, preferably an aqueous carrier. A variety of aqueous excipients may be used, e.g., water, buffered water, 0.4% saline, 0.3% glycine, hyaluronic acid, and the like. These compositions may be sterilized by conventional, well known sterilization techniques, or may be sterile filtered. The resulting aqueous pharmaceutical compositions may be packaged for use as is, or lyophilized, the lyophilized preparation being combined with a sterile solution prior to administration. The pharmaceutical compositions may contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, among many others. Thus, a typical pharmaceutical composition for intradermal infusion could be made up to contain 250 mL of sterile Ringer's solution, and 100 mg of peptide. Actual methods for preparing parenterally administrable pharmaceutical compounds will be known or apparent to those skilled in the art and are described in more detail in for example, Remington: The Science and Practice of Pharmacy (“Remington's Pharmaceutical Sciences”) Gennaro A R ed. 20th edition, 2000: Williams & Wilkins PA, USA, which is incorporated herein by reference.

The route and regimen of administration will vary depending upon the population and the indication for vaccination, and is to be determined by the skilled practitioner. For example, the immunostimulatory peptides, including their functional variants, and the peptide-containing compositions of the present disclosure can be administered in such oral dosage forms for example as tablets, capsules (each including timed release and sustained release formulations), pills, powders, granules, elixirs, tinctures, solutions, suspensions, syrups and emulsions, or by injection. Similarly, they may also be administered parentally, e.g., in intravenous (either by bolus or infusion methods), intraperitoneal, subcutaneous, topical with or without occlusion, or intramuscular form. In some embodiments, the peptides and peptide-containing compositions are administered intradermally or subcutaneously. In some embodiments, the peptides and peptide-containing compositions are intranasally. All of these forms are well known to those of ordinary skill in the pharmaceutical arts.

The vaccination dose of the peptides and the pharmaceutical compositions of the present disclosure may be varied over a range from about 0.001 to about 1,000 mg per adult per vaccine or treatment dose. For oral administration, the compositions are preferably provided in the form of tablets containing from about 0.001 to 1,000 mg, preferably about 0.001, 0.01, 0.05, 0.1, 0.5, 1.0, 2.5, 10.0, 20.0, 50.0, and 100.0 milligrams of the one or more immunostimulatory polypeptides. An effective amount of the pharmaceutical composition comprising the immunostimulatory polypeptide or more than one immunostimulatory polypeptides is ordinarily supplied at a dosage level of from about 0.0001 mg/kg to about 50 mg/kg of body weight per inoculation. The range is more particular from about 0.0001 mg/kg to about 7 mg/kg of body weight per dose. Doses for parenteral administration of the pharmaceutical composition comprising at least one immunostimulatory polypeptide described herein can range from 0.0001 mg/kg to about 7 mg/kg of body weight per vaccine dose.

In some embodiments, the peptides of the present disclosure and/or their functional variants can be administered by injection to a subject in the form of a peptide-based vaccine. Preferably, the peptides are injected intradermally, subcutaneously, or intramuscularly to allow for uptake by or exposure to antigen presenting cells located in the skin, epidermis or dermis, although other routes of administration known in the art may be equally suitable and are intended to be included in the present disclosure. In some embodiments, the peptides of the present disclosure can be loaded, by encapsulation or surface attachment, onto virosomes, liposomes, or nanoparticles prior to administration to the subject, as described above. The peptide loaded delivery vehicles can then be injected into the subject via intradermal, subcutaneous or other suitable routes analogous to the administration of the peptides described previously. Advantageously, suitable formulations of the present disclosure may be administered in a single daily dose, or the total daily dosage may be administered in divided doses for example of two, three, or four times daily. The peptides, including functional variants and compositions of the present disclosure, may be used to prepare a medicament or agent. Furthermore, the immunostimulatory compositions of the present disclosure, particularly those containing virosomes or liposomes, can be administered in intranasal form or via transdermal routes known to those of ordinary skill in the art.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

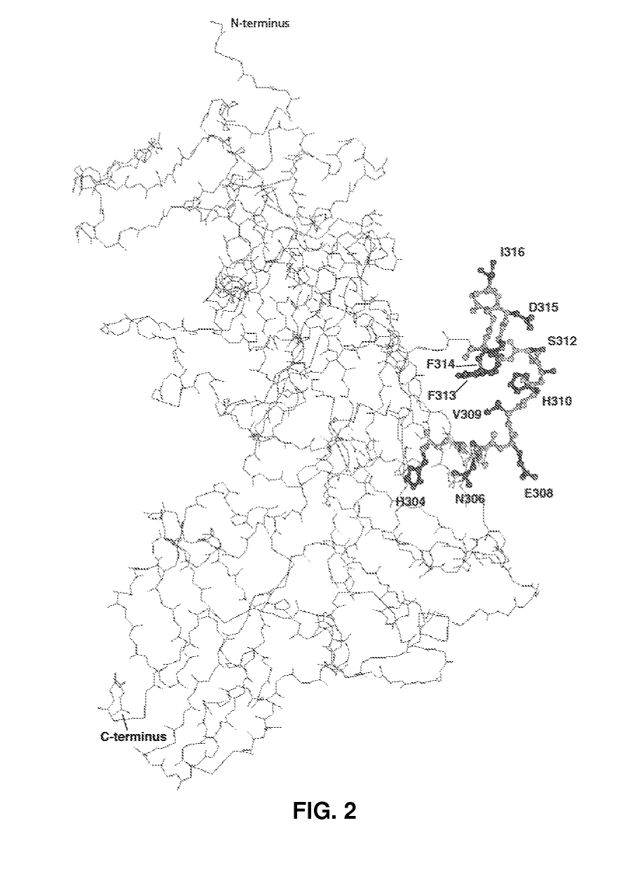

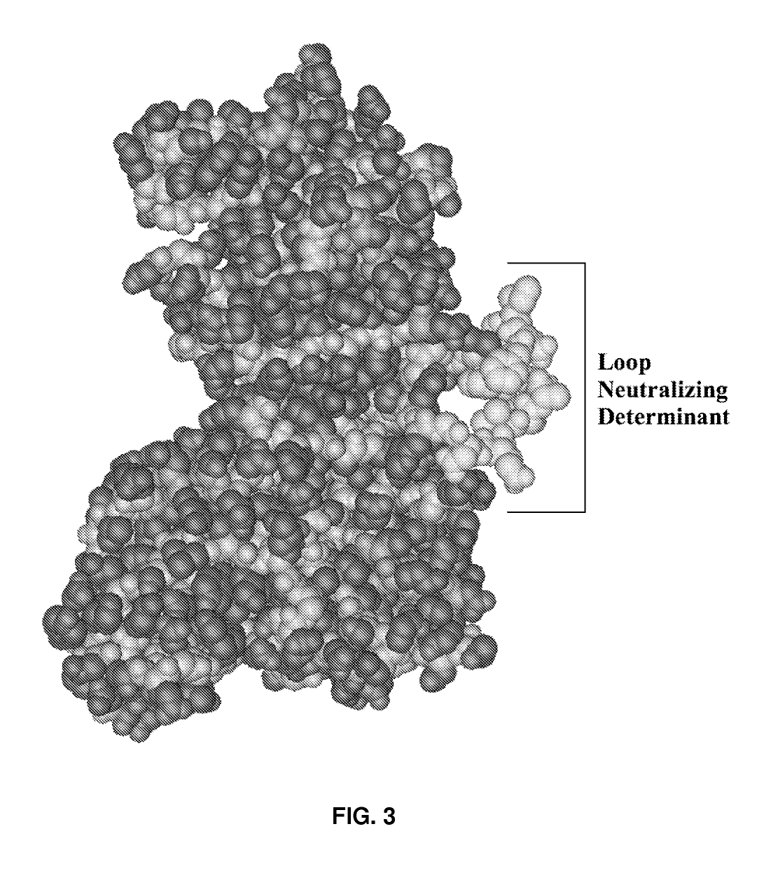

Linear Synthetic Peptide Vaccines. A panel of immunostimulatory polypeptides and T-B peptides targeting discrete regions on PA and LF were designed utilizing the analysis of the 1AAC crystal structure. A non-resolving loop in PA was later resolved with the publication of the 1TZN crystal structure. The new crystal structure (FIGS. 2 and 3) revealed the confirmation of the 2β2-2β3 loop as it exists in the PA heptamer and represented a potentially strategic sequence for targeting using a sub-unit vaccine.

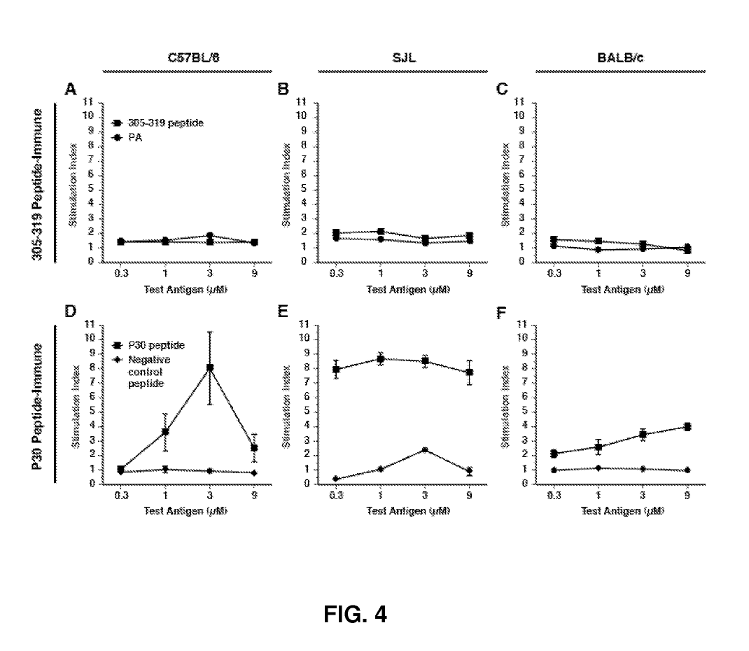

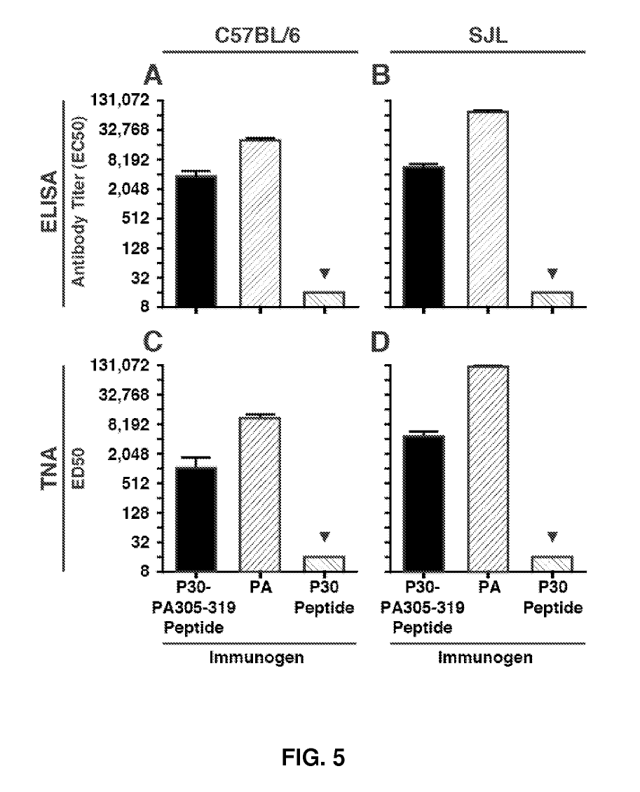

A structural-activity analysis of this newly resolved loop in domain 2 of PA focused on a.a. 305-319 as a target of humoral immunity. First, it was experimentally established that the 305-319 peptide was a hapten, devoid of the ability to stimulate helper T lymphocyte responses, in several distinct, genetically well-defined strains of mice: C57BL/6, SJL and BALB/c. T cell proliferation assays demonstrated that this was the case as shown in FIG. 4 panels A, B, and C. The peptide sequence, therefore, required a covalently-linked source of helper T cell stimulation for the generation of antibody responses. The 305-319 peptide was then synthesized as a T-B peptide colinearly-linked to the P30 helper site which elicts a strong T cell reponse in these strains as shown in FIG. 4 panels D, E, and F. Having established that the C57BL/6 and SJL strains are strong responders to the P30 epitope, the P30-PA305-319 peptide (henceforth referred to as P30-PA305) was tested in C57BL/6 and SJL mice using Quil A adjuvant. As shown in FIG. 5 panels A and B, the C57BL/6 and SJL mice immunized with the P30-PA305 generated high-titered antibody that was reactive with immobilized PA by ELISA. As expected, whole PA was very immunogenic in the mice and elicited antibodies that also reacted with immobilized PA. P30 peptide alone induced P30-specific antibodies (not shown), but did not generate any antibodies cross-reactive with PA. Individual mouse sera were then assessed in the toxin neutralization assay (TNA). The assay assesses the ability of antibody to block LeTx action in vitro using the RAW264.7 cells as an indicator cell line, and is configured so that the final concentration of LeTx is about a 2.5× to 3.5× multiple of the amount needed to kill 50% (TD50) of the RAW264 cells.

As seen in FIG. 5 panel C and D, C57BL/6 and SJL mice immunized with the P30-PA305 peptide were capable of neutralizing LeTx in vitro at levels that were approximately 15% of the neutralization detected in the serum from mice immunized with soluble PA. Sera from mice immunized with the P30 peptide alone had no detectable neutralizing activity. The results demonstrated, for the first time, that immunization of mice with an epitope-specific immunogen could induce antibody capable of significant neutralization of LeTx in vitro. Similar results were obtained in additional studies in C57BL/6 mice using Alhydrogel and monophosphoryl lipid A adjuvants (MPL) and in SJL mice using both Quil A and a combination adjuvant containing both Alhydrogel and MPL adjuvant (not shown).

Having established that the P30-PA305 peptide elicits neutralizing antibody in the C57BL/6 and SJL mice, the immunogenicity of the T-B peptide was next assessed in rabbits, since the rabbit model is validated for use in evaluating all potential vaccines for anthrax. Seven rabbits were immunized with the P30-PA305 peptide in an emulsion with CFA for priming immunizations and IFA for booster immunizations.

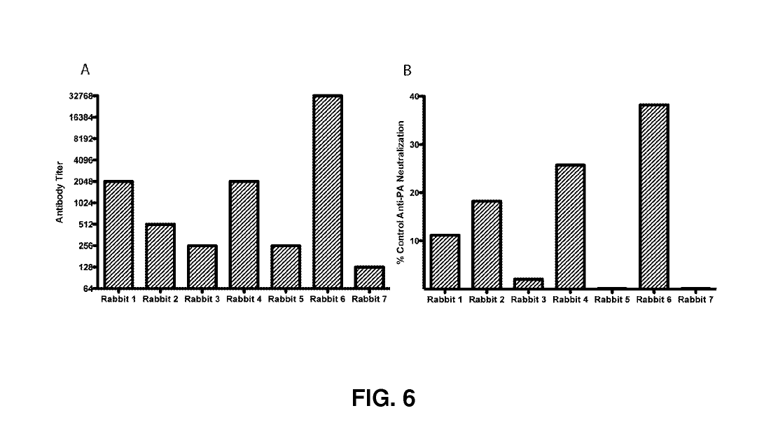

As seen in FIG. 6, despite the fact that the P30-PA305 peptide was not highly immunogenic in all rabbits, 5/7 rabbits were capable of neutralizing LeTx in vitro and 2/7 had TNA levels greater than 25% of the control anti-PA hyperimmune rabbit serum. The results corroborated the mouse data in demonstrating that the antibodies specific for the 305-319 region in domain 2 of PA are capable of neutralization. Further, the association of relatively low antibody titers among all but one rabbit with LeTx neutralization suggested that the PA305-specific antibodies exhibited potency. Also, the two rabbits exhibiting the highest neutralization also possessed the highest antibody titers, a trend also seen with the mouse data. This suggested that efforts to increase the magnitude of the antibody response might also lead to an increase in the levels of neutralization. The results in the mouse and rabbit are significant in that they identify a region of PA towards which epitope-specific LeTx-neutralizing antibody can be successfully induced using a sub-unit immunogen. Most importantly, they establish that the 305-319 site is a potentially important target for an epitope-specific vaccine against anthrax. This region is referred to as the loop neutralizing determinant (LND).

Though the levels of neutralization achieved in rabbits immune to the P30-PA305 peptide would not be capable of protecting rabbits in an anthrax spore inhalation challenge, the LND of PA appears to be a very important therapeutic and vaccine target against anthrax. Subsequent experimental efforts focused on increasing the magnitude and breadth of antibody responses in mice and rabbits towards the LND, and to furthering understanding of the significance of this region as a neutralizing site among the repertoire of neutralizing antibody specificities known to be induced through immunization with PA.

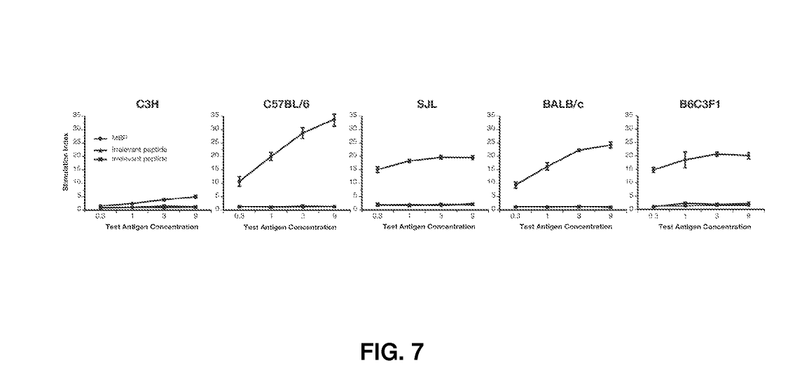

A Recombinant Tandem Repeat Immunogen Targeting the LND. Despite the effectiveness of the T-B peptide containing the P30 epitope for eliciting humoral immunity towards the LND, the peptides were not capable of stimulating immunity in all vaccinated rabbits and likely suffer from restricted helper T cell activity when used in an outbred population of animals. Indeed, the peptides were initially designed as probes for identifying neutralizing epitopes and were not expected to yield vaccine levels of immunogenicity. Therefore, a recombinant immunostimulatory polypeptide was engineered containing two tandemly repeated copies of the neutralizing epitope from domain 2 of PA, a.a. 305-319, covalently linked to 3 tandemly repeated copies of the Schistosoma mansoni P38P4 epitope and to maltose binding protein, with the intention of creating an immunostimulatory polypeptide with adequate sources of helper T cell epitopes to induce antibody production across multiple H-2 haplotypes of mice and in outbred rabbits. A helper cassette was developed for use in the recombinant immunostimulatory polypeptides containing the P38P4 epitope, (Shistosoma mansoni 38 kDa Soluble Egg Antigen 235-249; henceforth referred to as SM) from Shistisoma mansoni. This well-characterized epitope is known to be stimulatory in the C3H background. Since the MBP fusion protein linked to the recombinant immunostimulatory polypeptides lacks the ability to stimulate T cells in the C3H strain (FIG. 7), this epitope was a strategic choice in an effort to increase the likely breadth of helper T cell response to the recombinant constructs.

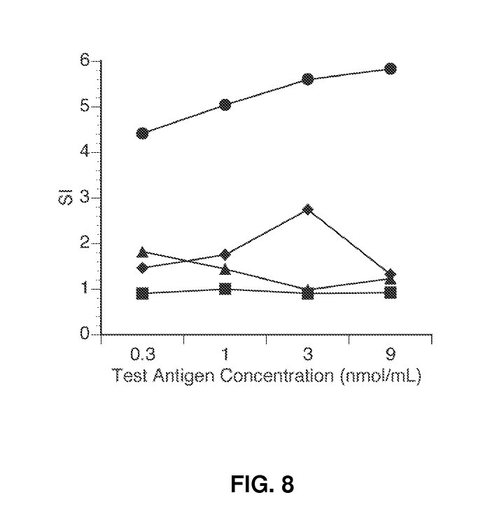

Before incorporating multiple copies of this sequence into the vector, the ability of the SM peptide to stimulate T cells in the C3H strain (FIG. 8) was confirmed. Then, the SM(3)PA305(2) immunostimulatory polypeptide, which contains 3 copies of the SM helper T cell epitope covalently linked to two copies of the PA305-319 sequence, was constructed.

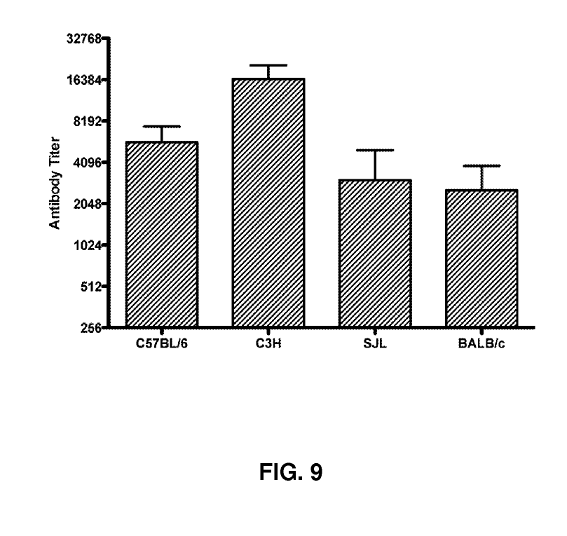

After confirming the fidelity of the recombinant immunostimulatory polypeptide DNA sequence and the quality of the purified immunogen, the immunogenicity of the SM(3)PA305(2) immunostimulatory polypeptide was tested in four strains of mice. FIG. 9 shows the geometric mean titers from 4 strains of mice immunized with SM(3)PA305(2). As shown in FIG. 9, immunization of all four strains of mice tested was successful in inducing antibody that cross-reacted with PA by ELISA. The highest titers observed were in the C3H strain where the SM helper T cell epitope would be expected to exert a pronounced effect. The SM(3)PA305(2) was also found to be highly immunogenic in F1 mice (B6C3F1, not shown). The murine antiserum was also capable of neutralizing LeTx in vitro (not shown).

The SM(3)PA305(2) immunostimulatory polypeptide was then evaluated in four outbred rabbits using CFA for priming immunizations and IFA for booster immunizations. Rabbits were immunized at two-week intervals with the SM(3)PA305(2) immunostimulatory polypeptide using CFA/IFA adjuvant and serum was analyzed for antibody and TNA titers.

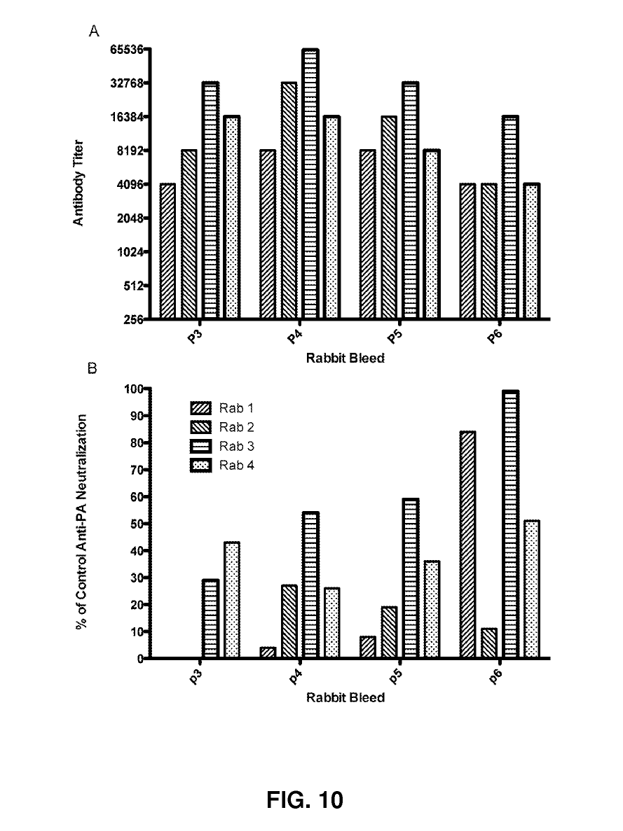

As seen in FIG. 10, all rabbits developed antibody and neutralization titers, with the peak neutralization observed following the 6^{th} immunization in all but one rabbit (rabbit #2). Three out four rabbits exhibited neutralization levels at or exceeding 50% of the control anti-PA neutralization with 2/5 rabbits exceeding 80% of control. Despite the peak titers occurring following the 4^{th} immunization, it appears that the maturation of the antibody response, and likely, the maturation of the antibody affinity, results in the progressive increase in the levels of neutralization to the peak levels observed following immunization #6.

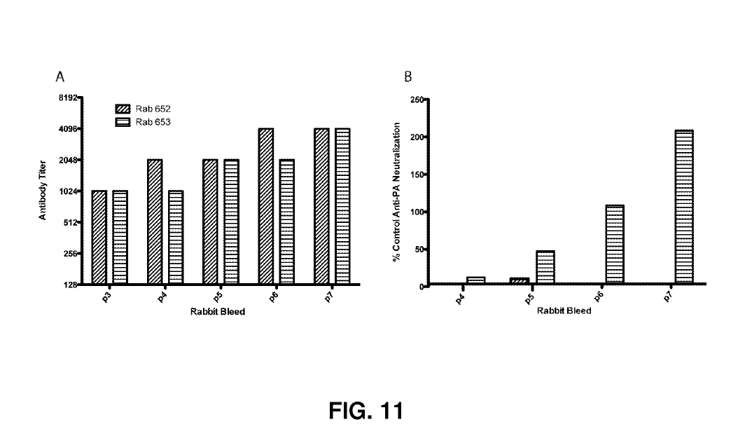

The performance of the recombinant immunostimulatory polypeptide in the outbred rabbits led to evaluating the SM(3)PA305(2) in rabbits using Alhydrogel with monophosphoryl Lipid A (MPL), an adjuvant quite a bit weaker than Freund's adjuvant but one being considered for human use. In this experiment, two rabbits were immunized with the SM(3)PA305(2) immunostimulatory polypeptide every two weeks and serum was collected 10 days after injections 3-6. FIG. 11 shows a time course of serum antibody and neutralization titers from the two immunized rabbits. Though both rabbits reached half maximal reciprocal titers of 4000, only one rabbit had antibody capable of neutralizing LeTx in vitro. Somewhat strikingly, however, despite the relatively low titer, rabbit 653 neutralized LeTx at levels that were about 200% of the neutralization observed in the control anti-PA serum from rabbits hyperimmune to soluble PA, confirming earlier observations with rabbits immune to the P30-PA305 peptide that antibody specific for the PA305-319 can exhibit striking potency. As shown, rabbit 652 had the equivalent antibody titer to rabbit 653, yet serum from this rabbit exhibited only minor LeTx neutralization in vitro following the 5^{th} immunization and was otherwise incapable of LeTx neutralization. The two rabbits, therefore, whose serum contained dramatically different levels of neutralizing antibodies, were indistinguishable with the current solid-phase immunoassay.

In summary, the SM(3)PA305-319(2) recombinant immunostimulatory polypeptide was found to be capable of eliciting high-titered humoral responses in 4 inbred strains of mice and an F1 hybrid. The murine antiserum cross-reacted with whole PA by ELISA and was capable of neutralizing Bacillus anthracis lethal toxin in vitro. Subsequent immunization of 4 outbred rabbits with the SM(3)PA305-319 tandem repeat immunostimulatory polypeptide in an emulsion with Freund's adjuvant yielded high titered antibody responses which were capable of LeTx neutralization in vitro. Three out of four rabbits had neutralizing responses that were 50% or greater of the control anti-PA antiserum, which is considered the gold-standard for Bacillus anthracis immunity, and two of the rabbits exceeded 80% of control. Finally, one out of two rabbits immunized with the recombinant immunostimulatory polypeptide in an Alhydrogel/MPL adjuvant had neutralizing titers that were 200% of the control anti-PA antiserum. It is concluded that the SM(3)PA305(2) tandem repeat immunostimulatory polypeptide represents a vaccine for Bacillus anthracis.

Multiple Antigenic Peptides Targeting the LND. In parallel fashion to the development of the recombinant tandem repeat immunostimulatory polypeptides targeting the LND, a multiple antigenic peptide (MAP) displaying the PA 305-319 region was evaluated. MAPs have demonstrated ability to elicit high-titered responses to peptide sequences. In evaluating the immunogenicity of the MAP construct displaying the PA 305-319 peptide, a simple MAP containing the LND peptide without a covalently-linked helper sequence was tested first.

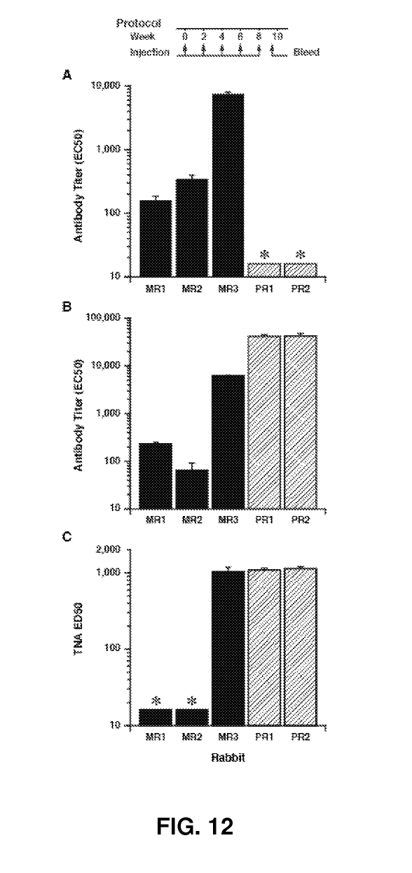

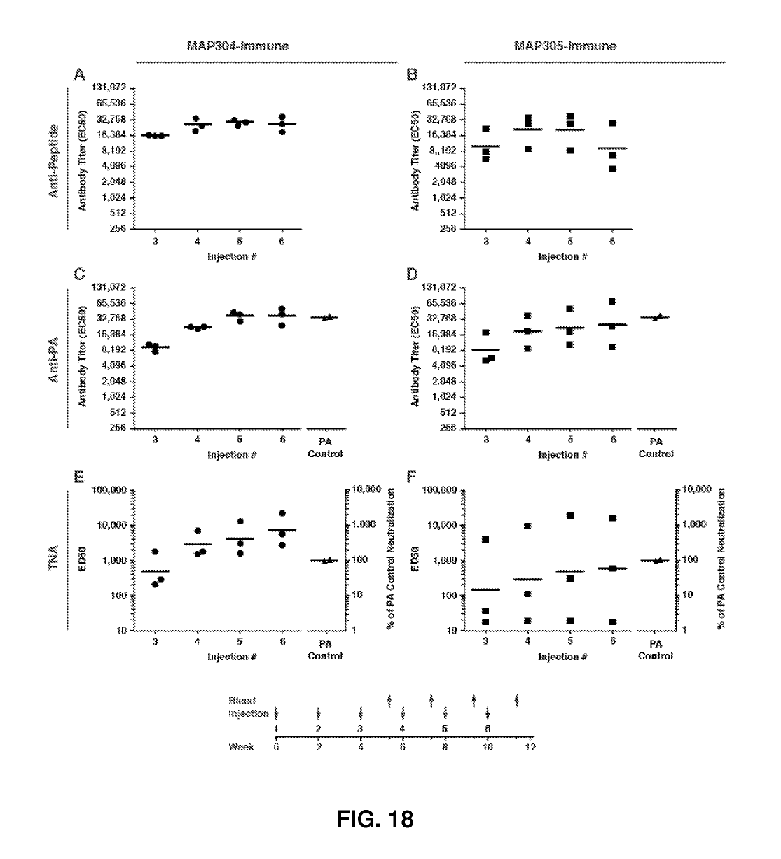

To evaluate whether antibodies could be elicited with specificity towards sequences within the 2β2-2β3 loop sequence of PA, a MAP peptide is synthesized displaying four copies per molecule of a.a. 305-319 from PA, and its immunogenicity assessed in rabbits, the species used most frequently to evaluate the protective efficacy of new anthrax vaccines. Three rabbits were immunized with the MAP peptide in an emulsion with CFA and boosted four times at two week intervals in an emulsion with IFA. As positive controls, two rabbits were immunized with full-length PA using the same adjuvant regimen. Rabbits were bled 10 days after the final immunization and sera was evaluated by ELISA for the induction of antibodies immunoreactive with the 305-319 peptide sequence and with PA. One of the three rabbits immunized with the MAP peptide developed a significant antibody response which bound both the peptide sequence and PA (FIGS. 12A and 12B, respectively). As expected, both rabbits immunized with full length PA developed high-titer PA-specific antibody responses (FIG. 18B).

It was next evaluated whether antibodies from the serum of the MAP-responder rabbit had activity in the TNA. The assay evaluates the functional ability of antibody to neutralize LeTx in vitro and TNA titers have been shown to correlate well with protection in inhalation spore challenges. The MAP-immune serum exhibited neutralization of LeTx at levels equivalent to the neutralization in the serum of the two positive control rabbits, even though the PA-specific antibody titer in the serum of this MAP-immune rabbit was less than 15% of that of the control PA-immune rabbits (FIG. 12C). This data suggested that antibodies to the 2β2-2β3 LND possess neutralizing potency exceeding that of PA antiserum.

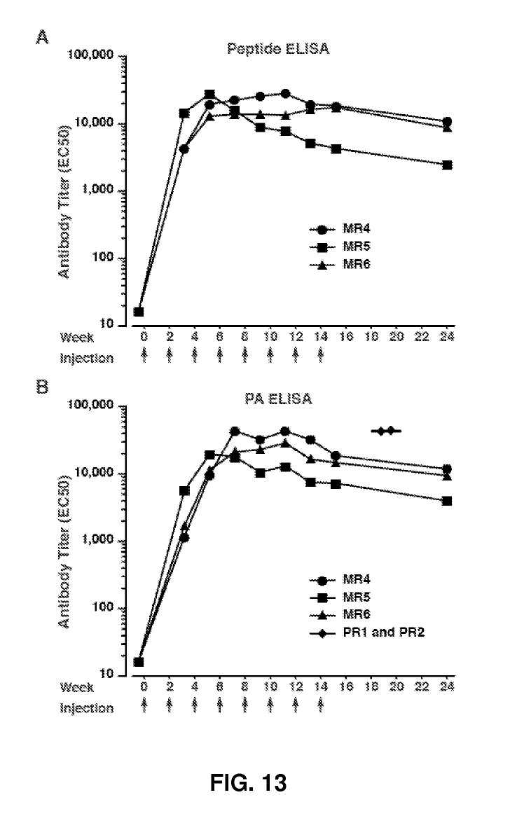

Having demonstrated that antibody to the LND mediated significant neutralization, an additional group of 3 rabbits was evaluated. Excepting a slight dose modification and additional booster immunizations, the second group of rabbits was immunized with the MAP peptide in identical fashion to the first group. As shown in FIG. 13A, all three rabbits from the second cohort developed significant anti-peptide titers as early as after the second injection (week 2). Near peak titers were maintained in rabbits MR4 and MR6 through the 8^{th} injection (week 14), while rabbit MR5 had anti-peptide titers which peaked early and then declined more rapidly. When assessed 2.5 months after the last injection, significant anti-peptide titers were observed in all three rabbits. Importantly, the antibodies were also found to be immunoreactive with immobilized PA (FIG. 13B). As with the anti-peptide titers, peak anti-PA titers appeared promptly, and were maintained through injection 8 in rabbits MR4 and MR6. Thereafter, anti-PA titers dropped slightly, but remained significant at the time of the terminal bleed, 2.5 months after the final booster immunization, with reciprocal EC50 titers of 11,857, 9,447 and 3,990.

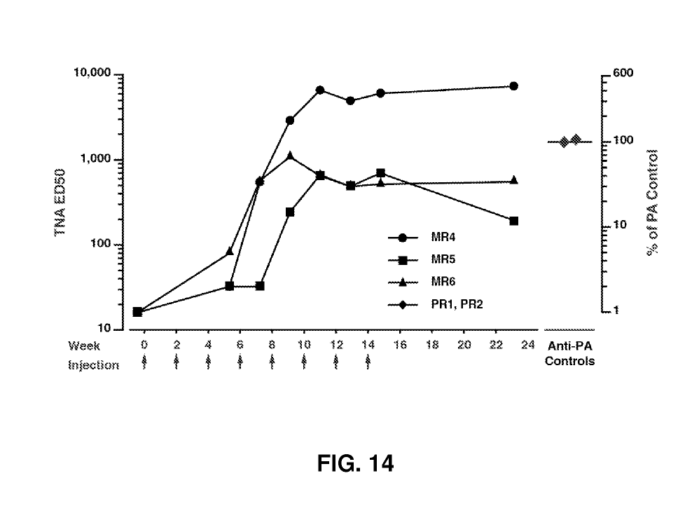

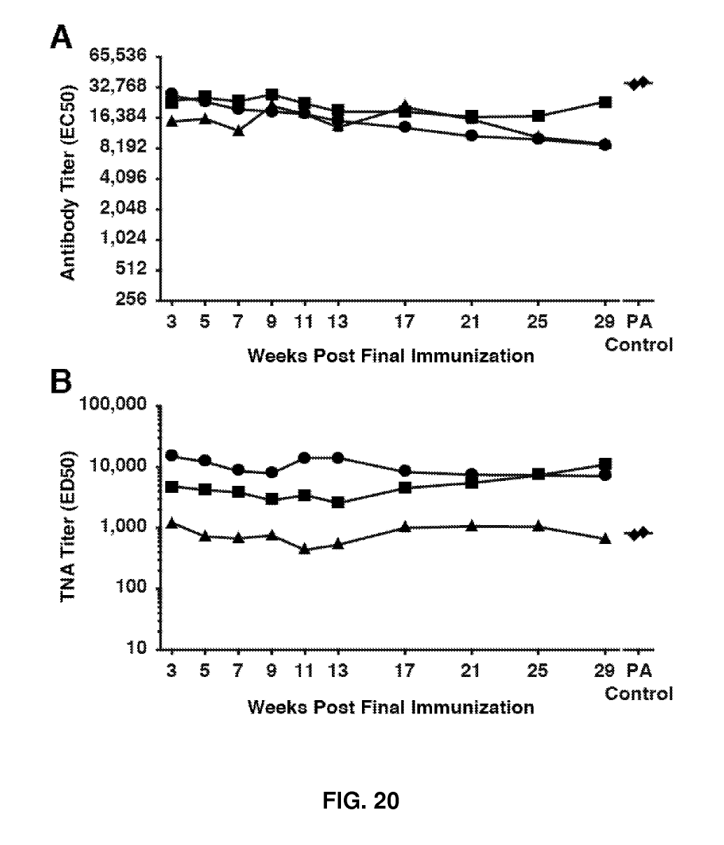

Neutralization analysis of the expanded cohort showed that all three MAP-immune rabbit antisera exhibited high-titer neutralization of LeTx in vitro (FIG. 14). At their peak levels, rabbit MR4 demonstrated neutralization that was over 450% of the mean neutralization from the two positive control PA-immune rabbits, with the other two MAP-immune rabbits, MR5 and MR6, demonstrating peak neutralization titers which were 41% and 67% of the positive control neutralization, respectively. Unlike the kinetics of antibody induction, neutralization peaked later, occurring between the 5^{th} and 6^{th} immunization (week 8 and week 10, respectively) for rabbits MR5 and MR6, and at the final bleed for rabbit MR4, 2.5 months after the final immunization. At this final time point, serum from the three rabbits demonstrated neutralization levels that were 452%, 34% and 12% of the two PA-immune control rabbits (FIG. 20).

Assessment of antibody avidity in MAP and PA-immune rabbit serum. In vitro and in vivo neutralization of virus and toxin have been shown to correlate well with the presence of highly avid antigen-specific antibodies. Observing that the levels of neutralization evident in the serum of the MAP-immune rabbits was quite high relative to the levels of antibody (i.e., potent), the progression of PA-specific avidity maturation in the sera of the second group of MAP-immune rabbits using an NH₄SCN chaotrope-elution ELISA was characterized. In this ELISA, the resistance to disruption of the antigen-antibody interaction in the presence of a fixed concentration of chaotrope is directly correlated with antibody avidity.

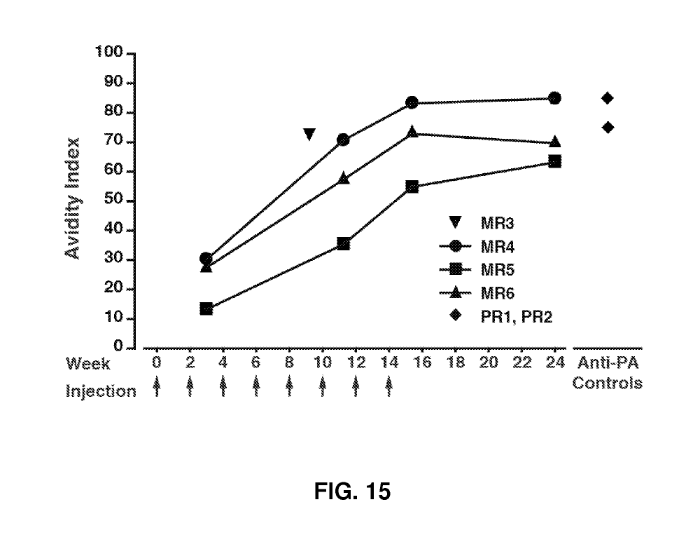

The development of antibody avidity was found to trail the development of antibody titer, peaking considerably later, as has been noted in other studies evaluating avidity maturation in human and murine models. Avidity rose over time, peaking in rabbits MR4 and MR5 at the time of the terminal bleed, 2.5 months after the final immunization, and in rabbit MR6, after the final immunization (FIG. 15). Antiserum from rabbit MR3, which like rabbit MR4, had neutralization equivalent to or greater than the neutralization observed in the PA controls, also demonstrated highly avid antibody, as did the polyclonal antiserum from the two PA-immune control rabbits.

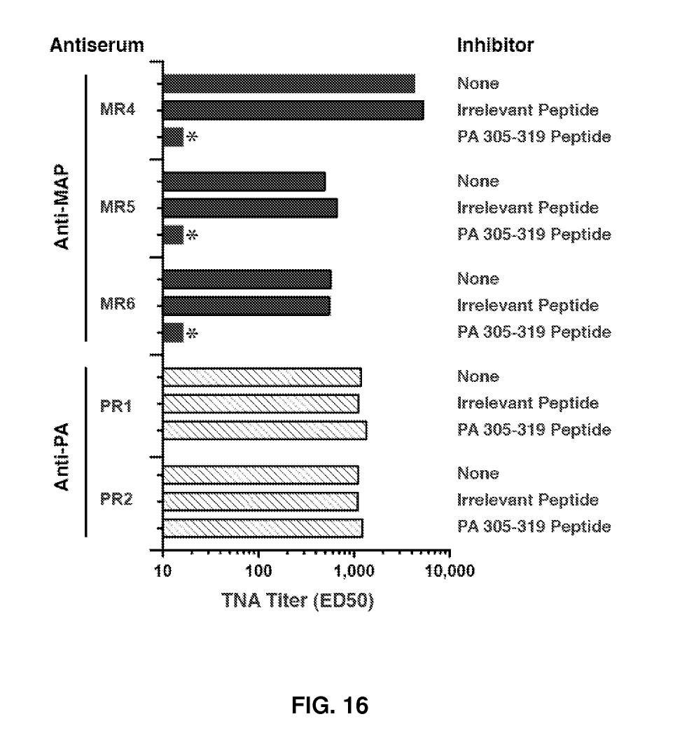

The neutralizing activity in the serum of MAP-immune rabbits can be completely inhibited with peptide. To assess whether the neutralizing antibodies in the sera of the MAP-immune rabbits were specific for, and whether they can be inhibited, a linear sequence within the 2β2-2β3 loop of PA, antisera from the second group of MAP-immune rabbits, and the sera from the PA-immune controls, were pre-incubated with the 305-319 peptide prior to assessment in the TNA. All neutralizing activity in the sera of the MAP-immune rabbits was completely inhibited when sera were pre-incubated with the 305-319 peptide (FIG. 16). This inhibition was specific, as no inhibition was detected when the antisera was incubated with an irrelevant peptide. As shown, there was no detectable inhibition in the positive control sera from the rabbits immunized with PA, suggesting that the LND neutralizing specificity was not present at meaningful levels in the PA antisera.

In summary, a MAP peptide, displaying 4 copies per molecule of the 305-319 peptide sequence from the 2β2-2β3 loop of PA, was evaluated for the ability to stimulate epitope-specific humoral immunity in rabbits. The 2β2-2β3 loop of PA presents a target for an epitope-specific vaccine for anthrax. Unresolved in the crystal structure of monomeric PA (1ACC and 1T6B), the 2β2-2β3 loop was first revealed with the crystallization and solution of heptameric PA and CMG2. The loop has been shown to be critical for translocation of LF and EF into the cytosol and contains the chymotrypsin cleavage site, which must be intact for LeTx cytotoxicity. It follows that antibodies specific for this site may interfere with critical molecular interactions. Overall, 4 out of 6 rabbits immunized with the MAP peptide developed high titer, high avidity antibodies, which were immunoreactive with immobilized peptide and PA, and which exhibited high levels of LeTx neutralization. Significant durability of neutralization was also observed, as reflected in the increase in neutralization titers for rabbits MR4 and MR6 from their 8^{th} and final injection, to their terminal bleeds 2.5 months later. The role of Freund's adjuvant, a water-in-oil emulsion, was considered in explaining the results; however, this effect in rabbits immunized with PA in Freund's adjuvant is not seen, where more typically, it is observed that a significant diminution in LeTx neutralization occurs by 4-6 weeks after the final immunization, a pattern also seen with rabbits immunized with PA using other adjuvants. The evidence, instead, shows that the durability results from the use of the MAP peptide, a form of immunogen which has been shown to be capable of eliciting highly durable antibody responses.

The neutralization titers among the MAP-immune responder rabbits compared favorably with the titers observed in the positive control rabbits immunized with PA in Freund's. One MAP-immune rabbit, in particular, had neutralization titers that were greater than 450% of the mean neutralization observed in the serum of the control PA-immune rabbits, indicating that antibody to this neutralizing determinant can exhibit significant potency. This rabbit also demonstrated the highest antibody titers to immobilized PA among the MAP-immune responder rabbits (FIG. 13B). Among the three other responder rabbits, peak levels of neutralization were 100%, 67% and 41% of the PA-immune control rabbits. Though neutralization is considered the best in vitro surrogate for vaccine efficacy in aerosol spore challenges in rabbits, there is overlap in the ranges of neutralization titers which are associated with survivors and non-survivors. Nevertheless, PA-specific responses with neutralization titers exceeding 750 to 1,000 appear most predictive of protection. Using such criteria, three out of four MAP-immune responder rabbits would be predicted to survive a B. anthracis spore challenge if performed after their 5^{th} immunization. More importantly, the data suggests that effective targeting of this epitope, whether using a totally synthetic MAP peptide as outlined herein, or one or more variant immunogens, could lead to meaningful and potentially protective levels of epitope-specific immunity against anthrax.

Although the current MAP peptide targeting the LND elicited neutralizing responses in the majority of rabbits, two MAP-immune rabbits were non-responders, presumably due to a lack of T cell help. Data in mice showed that the 305-319 peptide sequence is haptenic, or devoid of intrinsic helper T cell epitopes (FIG. 4A-4C). Since the MAP construct was not synthesized containing exogenous helper T cell sequences, the helper T cell stimulation necessary for the induction of antibody to the 305-319 sequence appears principally derived from neodeterminant sequences within the MAP construct. Therefore, the immunity towards the LND may be significantly enhanced, and MHC-restricted non-responsiveness minimized, by incorporating helper T cell epitopes into MAP immunogens and variants thereof that include the LND.

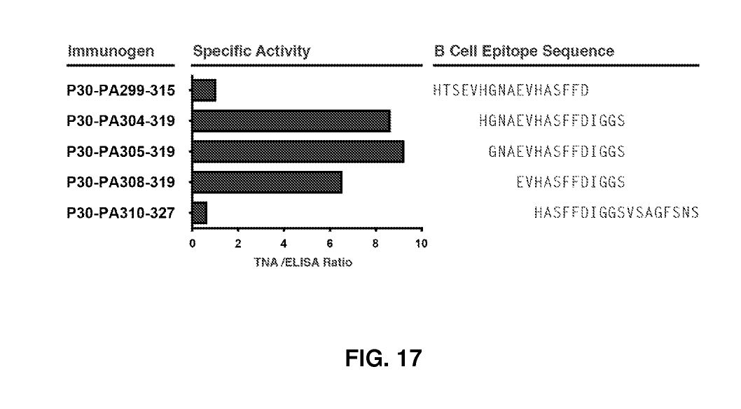

Prior to evaluating the addition of helper T cell epitopes to the MAP constructs, we first more precisely defined the optimal LND peptide sequence comprising the LND antibody target. A panel of linear peptides was synthesized representing sequences both N- and C-terminal to a.a. 305-319 from 2β2-2β3 loop of PA, all colinear with the P30 helper sequence at their N-terminus. Separate groups of B6C3F1 mice (H-2^{b}×H-2^{k}), a strain experimentally confirmed to be responders to the P30 epitope (not shown), were then immunized four times at two week intervals with one of the T-B peptides, and antisera were evaluated by ELISA and TNA. FIG. 17 demonstrates the specific activity, operationally defined as the quotient of the ED₅₀ TNA titers and the EC₅₀ antibody titers, associated with the group-specific antisera from mice immunized with each of the T-B peptides. As shown, the peptide sequences associated with the highest specific activity were focused within the region delineated by a.a. 304-319 of PA, with little activity observed in the serum of mice immunized with the peptides comprising sequences within the N- and C-terminal portions of the 2β2-2β3 loop region studied.

The optimized LND sequences were then incorporated into multiple antigenic T-B peptides for testing in rabbits. Two MAPs, each displaying 4 copies per molecule of either a.a. 304-319 or a.a. 305-319, each colinearly synthesized with the P30 helper epitope at the N-terminus, were then evaluated. Three rabbits per group were immunized five times at two-week intervals with the respective MAP construct in emulsions with CFA for priming immunizations and IFA for booster immunizations. Both MAPs were highly immunogenic, rapidly inducing antibody responses that were immunoreactive with both immobilized LND peptide sequence and with PA (FIG. 18A-18D). Serum antibody responses from the MAP304-immune rabbits displayed less variability and were more consistently elevated compared to the responses seen in the MAP305 rabbits, and achieved PA-specific Ab titers by their fifth immunization equivalent to the polyclonal PA-specific Ab responses from the control PA antisera (FIGS. 18C and 18D).

In the TNA, sera from the MAP304 rabbits, in particular, were highly neutralizing with all rabbits demonstrating neutralization titers considerably exceeding the PA controls (FIG. 18E). One MAP304-immune rabbit and one MAP305-immune rabbit had peak neutralization titers that were 23- and 19-fold higher than the control PA antiserum, respectively, confirming observations that antibodies to the LND can demonstrate significant potency.

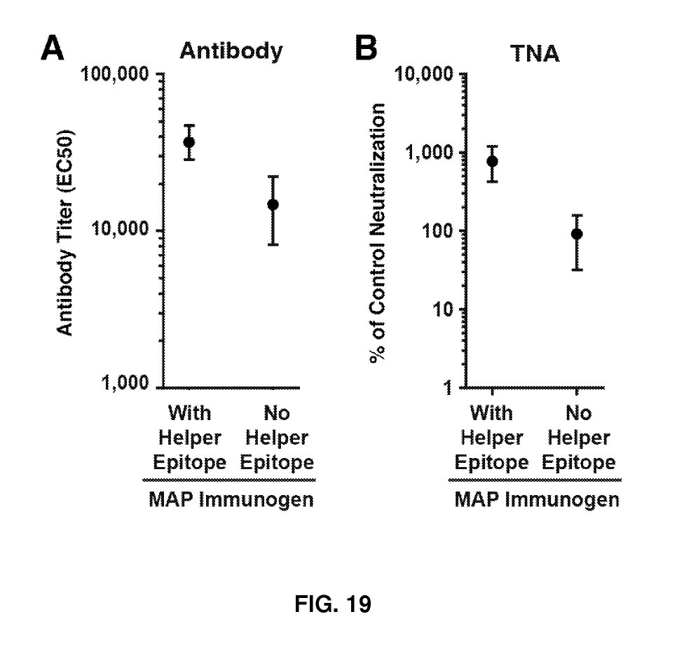

Antibody and TNA titers from the serum of the MAP304 and MAP305-immune rabbits obtained after injection 6 (week 12) were compared to the antibody and TNA titers from rabbits vaccinated in a prior study using the same immunization regimen, but with a MAP peptide displaying the 305-319 sequence without a helper epitope. The PA-specific antibody and TNA titers in the serum of the MAP304 and MAP305-immune rabbits were considerably higher than the titers in the sera of rabbits immunized with the MAP peptide without a helper epitope (FIG. 19A, 19B).

MAP304-immune rabbits display durable neutralizing responses. Excellent durability of antibody and neutralization titers was observed in rabbits immunized with a MAP peptide displaying 305-319 LND peptide but lacking a helper epitope. To assess the durability of the neutralizing responses from the three MAP304-immune rabbits, serial bleeds were obtained from the three rabbits following their 6^{th} and final immunization and evaluated the sera by ELISA and in the TNA. Excellent durability of both antibody and neutralization titers was evident in the antisera from all three rabbits, with little diminution of the TNA titers over the 7 months following the last immunization (FIG. 20A, 20B). At the final serum analysis, 29 weeks after the last immunization, two out of three MAP304-immune rabbits still demonstrated neutralization titers that were distinctly above the neutralization in the control PA antisera, and the third rabbit had neutralization levels only marginally below the neutralization titers of the control PA antiserum.

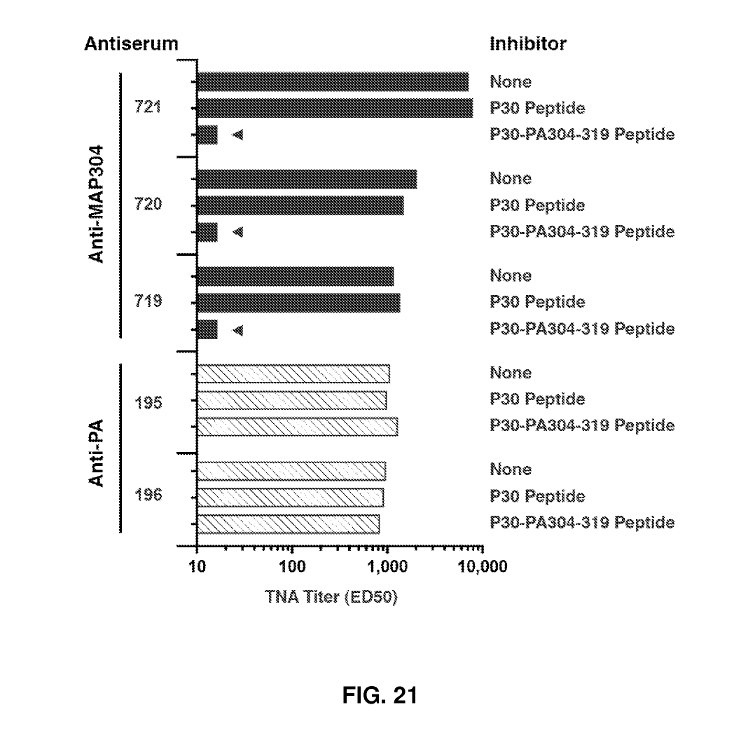

Determination of peptide-specific antibody affinity in the sera of MAP304 and MAP305-immune rabbits. The neutralization present in the serum of rabbits immunized with an LND-displaying MAP peptide can be completely inhibited with peptide. As shown in FIG. 21, TNA activity in the serum from the MAP304 rabbits is also completely and specifically inhibited with the P30-PA304-319 linear peptide but was unaffected by incubation with the P30 peptide alone. As expected, pre-incubation of control PA antisera with the P30-PA304-319 peptide had no effect on the neutralization titers of the control PA antisera.

Since the antibody responsible for the TNA activity in the sera of the MAP-immune rabbits was determined to be completely peptide-specific, the peptide-specific IC₅₀s, which are good approximations of the affinity (Kd) of the LND-specific antibodies present in the rabbit antisera, were determined. This allowed evaluation of: 1) whether differences exist in the peptide-specific affinity of antibodies in the sera of rabbits immunized with a MAP peptide containing the P30 helper epitope compared to those immunized without a helper epitope from a prior study, and 2) whether a correlation exists between affinity of the LND-specific antibody and neutralization.

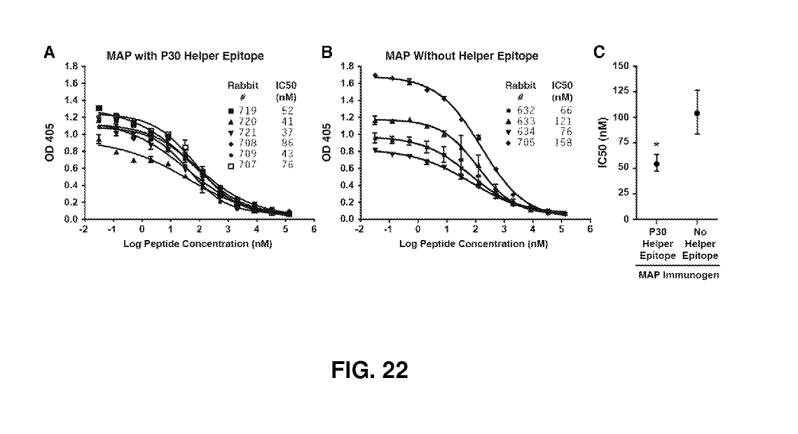

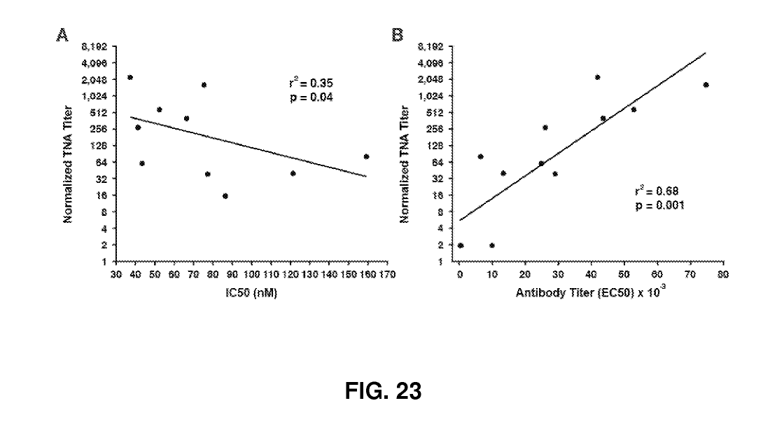

Sera from all six rabbits immunized with the MAP304 and MAP305 constructs were highly avid, with peptide-specific IC₅₀s (Kds) ranging from 37-86 nM and a mean IC₅₀ of 56 nM (FIG. 22A). Sera from the four helper-free MAP-responder rabbits from a previous study demonstrated significantly lower antibody affinity, with a mean IC₅₀ of 105 nM (p=0.019, FIGS. 22B, 22C). Peptide IC₅₀s from all of the MAP-immune rabbits from the current and previous study were found to be inversely correlated to TNA with a coefficient of determination (R²) of 0.35 (p=0.04, FIG. 23A). Antibody titer was found to be more highly correlated with TNA with an R² of 0.68 (p=0.001, FIG. 23B).

The present data have shown that peptide sequences in the 2β2-2β3 loop of PA provide a target for an epitope-specific vaccine for anthrax. The criticality of this site for toxin formation combined with the apparent absence of this potent specificity in the antibody repertoire elicited by whole PA, make it an attractive and strategic target for an epitope-specific vaccine for anthrax.

To summarize, the LND target was studied using engineered T-B peptides containing a broadly-restricted helper T cell epitope from tetanus toxin, P30, colinearly synthesized with a 15 or 16 a.a. haptenic peptide from the LND. In MAP form, the T-B peptides, as represented in the MAP304 and MAP305, were shown to elicit rapid and potent immunity to the LND, in some rabbits demonstrating LeTx neutralization orders of magnitude greater than the neutralization observed in positive control PA antisera from rabbits which had been immunized with PA in Freund's adjuvant. Though the antibody titers from the control PA antisera were essentially equivalent to the antibody titers in the serum of 5 out of 6 of the LND-immune rabbits when tested on immobilized PA (FIG. 18), the control PA titers reflect immunoreactivity to a multitude of PA epitopes, only some of which are neutralizing. Conversely, the LND-specific antibodies exhibit considerably greater neutralizing potency, since they are specific for a linear neutralizing epitope within the 2β2-2β3 loop, which the data in mice suggests resides between a.a. 304-319 of PA. The mapping to the peptide region comprising a.a. 304-319 is inclusive of the ³¹²SFFD³¹⁵ sequences found to represent the critical neutralizing determinant for several mAbs specific for this site.

When compared to the antibody and neutralization titers from sera of rabbits immunized with a MAP peptide displaying the 305-319 LND sequence without a helper epitope from a previous study, the MAP304 and MAP305 sera were found to exhibit considerably higher titers. The potentiation of hapten-specific humoral immunity through the use of helper T cell epitopes parallels work in other models but has not been shown previously with anthrax. The P30 helper T cell epitope has been shown to potentiate responses to covalently-linked B cell epitopes in both linear and MAP formats. The inclusion of the P30 helper T cell epitope in the MAP304 and MAP305 also reduced the incidence of MHC-restricted non-responsiveness, since all rabbits demonstrated significant antibody production, whereas 2 out of 6 rabbits in a previous study failed to produce meaningful levels of antibody when immunized with a MAP peptide displaying the 305-319 peptide without a helper epitope.

Though all six MAP304- and MAP305-immune rabbits developed significant antibody titers, one rabbit failed to develop meaningful levels of neutralization. The explanation for the poor neutralizing response of this one MAP305 rabbit is unclear, but since this rabbit had the lowest PA- and LND-specific antibody titer and LND-specific antibody affinity among all the MAP-immune rabbits, it may have been a low-responder to the P30 helper epitope. Among the five other MAP304 and MAP305 rabbits which did demonstrate neutralization, four would be expected to survive a lethal aerosol challenge with B. anthracis administered subsequent to the 4^{th} immunization and all five would be expected to survive a challenge administered after the 6^{th} immunization.

The neutralization observed in the MAP304-immune rabbits was found to be exceptionally durable, with TNA titers at 7 months after the last immunization still exceeding the PA control levels.

LND-specific antibody in the sera of the MAP304- and MAP305-immune rabbits was shown to have significantly higher affinity than the LND-specific antibody in the serum of rabbits immunized without a helper epitope. This result is consistent with the early seminal work with DNP, highlighting the critical role of cognate T cell help for the induction of high affinity antibody to a covalently-linked hapten. The increase in affinity of LND-specific antibodies in the sera of MAP304 and MAP305 rabbits may have contributed to the increased TNA titers observed in the serum of these animals compared to the neutralization observed in rabbits immunized with an LND MAP without a helper epitope.

A few studies examining mAbs specific for PA have demonstrated associations between antibody affinity and anthrax toxin neutralization. In the present study, antibody affinity was found to correlate with TNA titer, with an R² of 0.35. Not surprisingly, however, PA-specific antibody titer was shown to be more highly correlated to neutralization with an R² of 0.68. The correlations between peptide-specific antibody titer and neutralization determined from analysis of the MAP304 and MAP305 antisera are higher when evaluating reactivity to the 299-315 peptide sequence compared to the 304-319 peptide sequence (not shown). This data suggests that the minimal neutralizing epitope resides between a.a. 304-315.

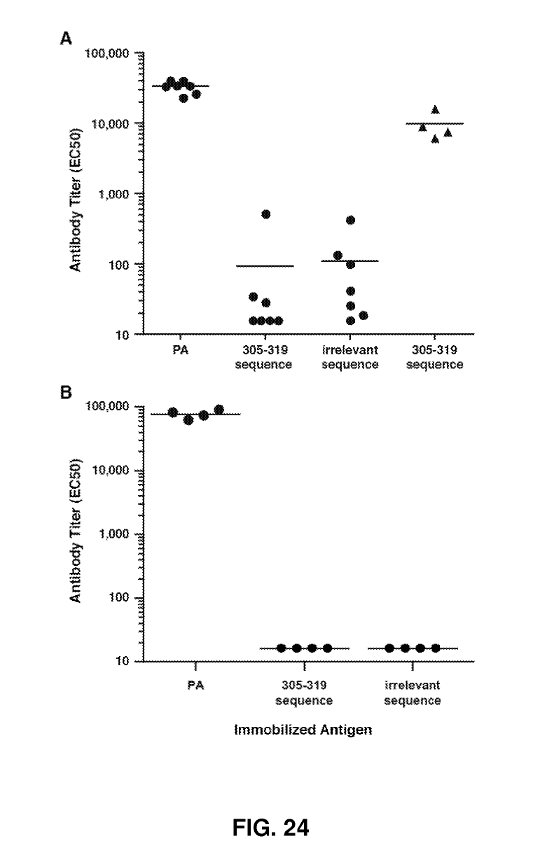

A totally synthetic LND vaccine for anthrax may find utility in a number of scenarios where the induction of rapid, high-titer neutralization is needed. These include post-exposure scenarios where the administration of a whole PA-containing vaccine may present safety concerns, in unvaccinated but high-risk populations, in individuals poorly responsive to AVA vaccination, and lastly, but perhaps most importantly, in scenarios where the neutralizing specificities stimulated by AVA might be intentionally subverted. PA-immune rabbits do not develop significant antibody to the LND. The absence of any detectable effect on neutralization when the 305-319 peptide was co-incubated with the control PA antiserum, combined with the finding that the PA antiserum lacks any immunoreactivity with immobilized 304-319 sequence by ELISA (FIG. 12A), led to assessment of whether PA-immune rabbits develop antibodies specific for the LND. To this end, 7 rabbits were immunized with PA in Freunds adjuvant and boosted two weeks later with PA in IFA. Rabbit antiserum was obtained 10 days after the booster immunization and was analyzed by ELISA for immunoreactivity with PA and with the 305-319 peptide sequence. All rabbits developed robust anti-PA responses with a reciprocal GMT of 32,696 (FIG. 24A). In contrast, the reactivity of the PA antiserum with the 305-319 peptide sequence was not significantly different from their reactivity with an irrelevant peptide sequence. As a control, sera from the four MAP-responder rabbits were shown to react strongly to the immobilized 305-319 peptide sequence. Antisera from the 7 PA-immune rabbits also were found to lack significant reactivity with a longer peptide sequence from domain 2 of PA which spanned a.a. 299-327 (not shown). The results demonstrate that there is no significant antibody reactivity with the LND in rabbits immunized with PA in Freund's adjuvant.

To further evaluate whether these results were specific only for the combination of PA and Freund's adjuvant, perhaps reflecting an interaction of the oil-in-water formulation on the presentation of PA, an additional cohort of 4 rabbits was immunized three times at two week intervals with PA in Alhydrogel®, the adjuvant used in AVA for humans. Rabbits were bled 10 days after the final immunization and antisera were evaluated by ELISA. Like the results observed with rabbits immunized with PA in Freund's, all rabbits developed high-titer responses reactive with immobilized PA with a GMT of 76,782; however, there was no reactivity with the 305-319 peptide sequence by ELISA (FIG. 24B).

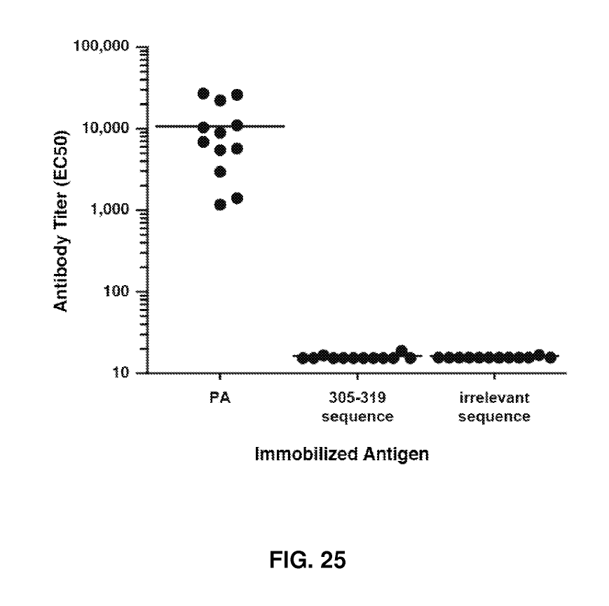

Finally, to more formally exclude the possibility that adjuvant formulations per se are responsible for the absence of LND-specific antibodies in PA-immune rabbits, antiserum from an additional series of 12 rabbits from a separate study was evaluated, which were previously inoculated with recombinant adeno-associated virus vectors expressing either PA63, or with vectors expressing both PA63 and LF. Though antiserum from all the rabbits had significant anti-PA titers, none of the rabbits demonstrated any reactivity to the LND peptide sequence by ELISA (FIG. 25).

The work of others suggests that guinea pigs immunized with PA also lack meaningful levels of LND-specific antibody. As described previously, the LND straddles the chymotrypsin cleavage site at ³¹³FFD³¹⁵ in domain 2 of PA, and both the present data and data from others evaluating mAbs to the LND, suggest that this region is a critical component of the neutralizing epitope. For example, Rhie et al. found no significant difference in the neutralization titers among guinea pigs immunized with either intact PA or chymotrypsin-cleaved PA, suggesting that the LND neutralizing specificity was not meaningfully represented among the repertoire of neutralizing antibodies in guinea pig PA antiserum (Rhie, G. E. et al. (2005) Efficacy of non-toxic deletion mutants of protective antigen from Bacillus anthracis. FEMS Immunol Med Microbiol 45:341-347.).

The diversity and relative abundance of neutralizing antibodies present in human AVA vaccine serum cannot be known with certainty. Nevertheless, current data from the isolation of mAbs in PA-immune mice and humans suggests a neutralizing antibody repertoire of limited specificity. Assays to evaluate the PA-specific neutralizing repertoire directly in the sera of AVA-immune primates have been explored, but a reliance on pre-defined mAb specificities constrains this approach. A limited neutralizing repertoire could represent a theoretical but real threat to the efficacy of vaccines that rely on the induction of PA-specific antibody.

Human vaccine antisera, like rabbit anti-PA antisera, appears to contain minimal LND-specific antibody. Gubbins et al. utilized a competitive enzyme-linked assay to evaluate whether AVR801(51), a human standard AVA-immune reference serum, and other individual AVA-immune human serum, could effectively compete with an LND-specific mAb (F20G75) for binding to PA. They found that only very high concentrations of AVA serum (half maximal inhibition at approximately neat dilutions) could inhibit the binding of the mAb to PA (Gubbins, M. J., L. Schmidt, R. S. Tsang, J. D. Berry, A. Kabani, and D. I. Stewart (2007) Development of a competitive enzyme linked immunosorbent assay to identify epitope specific antibodies in recipients of the U.S. licensed anthrax vaccine, Journal of immunoassay & immunochemistry 28:213-225). In contrast, AVA vaccinee serum demonstrates 100-fold more activity in inhibiting the binding of the 14B7 and 2D3 mAbs to PA, highlighting the significant presence in AVA serum of neutralizing specificities for the ATR and LF binding regions of PA, respectively (Reed, D. S., J. Smoll, P. Gibbs, and S. F. Little (2002) Mapping of antibody responses to the protective antigen of Bacillus anthracis by flow cytometric analysis, Cytometry 49:1-7).

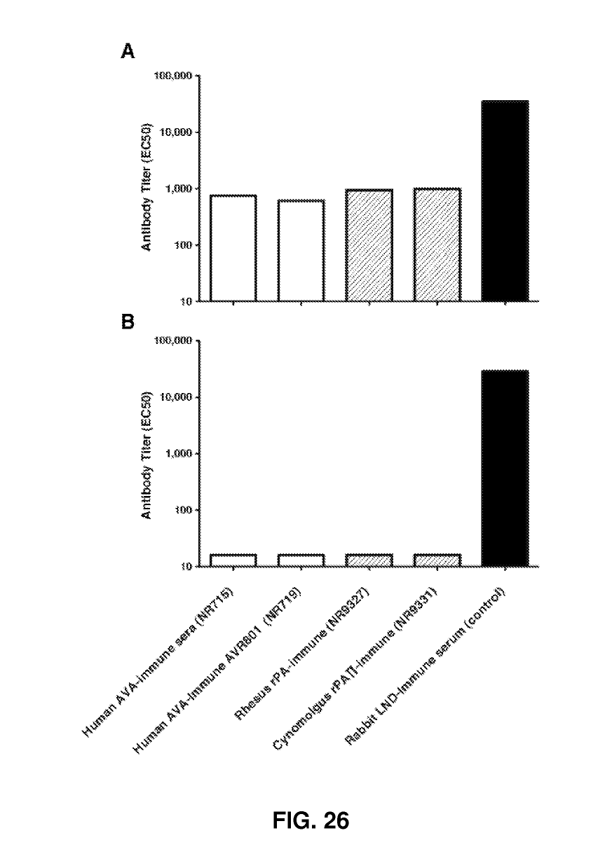

Human AVA sera and macaque rPA-immune sera were tested to determine whether they possessed antibodies specific for the LND peptide sequence. It was first established that the respective immune sera had antibodies reactive with PA. As shown in FIG. 26(A), all sera reacted with PA by ELISA with EC50 titers in the range of about 600-1000. As a control, a high titer rabbit anti-LND serum is also shown. The sera was then tested for reactivity with the LND peptide spanning a.a. 299-327 which encompasses the critical LND peptide sequences. As shown in FIG. 26(B), all of the human and macaque sera samples had no detectable antibody against the LND. The rabbit anti-LND serum is shown to react strongly with the 299-327 sequence. All samples were below the detection limit of the assay which is a reciprocal EC50 titer of 16. Thus, antibody specific for the LND is not present in the sera of non-human primates (NHP) immunized with recombinant protective antigen (rPA), where PA is the main immunogenic protective component of the current vaccine for anthrax, AVA. Also, the data highlights that sera from humans immunized with AVA also do not appear to have LND-specific antibody. These are two very important findings which impact the profile and importance of the present LND vaccines.

Collectively, these results demonstrate that the 2β2-2β3 loop region encompassing the LND behaves as if immunologically cryptic in many species. Though clearly antigenic in immobilized PA, as demonstrated through immunoreactivity by ELISA, and in solution, as reflected in its availability for protease cleavage by chymotrypsin, the 2β2-2β3 loop region encompassing the LND appears to be poorly immunogenic in PA.

The addition of the LND specificity to the current neutralizing specificities generated in primate PA antisera could improve the breadth of the neutralizing repertoire in ways important for countering potential bioterrorist threats. Analysis of mouse and human mAbs suggests that the ATR binding region in domain 4 is a dominant target for neutralizing antibody induced through immunization with PA. Experimental mutations in domain 4, however, have been shown to abrogate the effectiveness of 14B7, a well-studied and efficacious mAb with specificity for the ATR interface, while having little effect on the formation and toxicity of LeTx. This demonstration of evasion of an important neutralizing specificity, which is a constituent specificity of a potentially limited neutralizing repertoire, may suggest a vulnerability for PA-based vaccination strategies. These findings have prompted efforts to develop novel therapeutics designed to address such antibody resistant forms of LeTx. The LND neutralizing specificity targets the 2β2-2β3 loop region, which is critical for LeTx function, and the primacy of the loop sequences in enabling LeTx cytotoxicity may hinder or even preclude the malicious re-engineering of PA to evade the LND specificity. Other potential applications for a totally synthetic, epitope-specific anthrax vaccine targeting the LND may be for use in individuals who respond poorly to vaccination with PA-based vaccines, and possibly in post-exposure scenarios, where a role for vaccination is being studied, but where a reluctance to give vaccines containing whole PA might be warranted.

Efficacy Of LND-Targeted Immunogens For Protecting Against An Inhalation Spore Challenge With Aerosolized B. anthracis Ames Strain Spores. The efficacy of LND-targeted immunogens for protecting against an inhalation spore challenge with aerosolized B. anthracis Ames strain spores in the rabbit model was determined. Groups of 7 NZW female rabbits were immunized with the immunogens: rPA83 which is a recombinant full-length Protective Antigen (83 kDa.) (positive control); rMBP-SM3-(PA305-319)₂ which is a recombinant maltose binding protein fusion with three tandem copies of a helper T cell epitope from S. mansoni soluble egg antigen P38 (235-249), and two tandem copies of the 305-319 segment of B. anthracis PA representing the loop neutralizing determinant (LND); PA304-T*MAP which is a synthetic multiple antigenic peptide with four arms, each comprised of an N-terminal copy of the PA 304-319 LND B cell epitope, followed by a C-terminal copy of the T cell helper epitope from the P. falciparum CS protein (326-345), linked at its C-terminus to one of the four amino groups on the branching lysine core (B-T orientation); T cell-PA304MAP=a MAP with the alternatively ordered epitopes (T-B) such that an N-terminal copy of the T cell epitope is followed by a C-terminal copy of the PA 304-319 LND segment; P30-PA304MAP & T*-PA304MAP which are dual-helper-epitope combination immunogen containing: 1. a MAP with the P30 helper T cell epitope from C. tetani toxin (947-967) at the N-terminus followed by the PA 304-319 LND segment (T-B orientation), and 2. the T*-PA304MAP (T-B orientation) and P30-PA304MAP & PA304-T*MAP which are a dual-helper-epitope combination immunogen containing: 1. the P30 helper epitope-PA 304-319 LND T-B MAP, and 2. the PA304-T*MAP (B-T orientation). A naïve control included a group of 6 non-immune age-matched female NZW rabbits.

Animals were immunized with a priming immunization with antigen in complete Freunds adjuvant (CFA), followed by boosting immunizations with antigen in incomplete Freunds adjuvant (IFA) at weeks 3, 5, 7, and 9. At week 12, each rabbit received an inhalation challenge with a 200 LD50 target dose of aerosolized B. anthracis Ames strain spores in a randomized, blinded manner. Animals were observed over 14 days for death or pre-morbid debilitation requiring euthanasia.

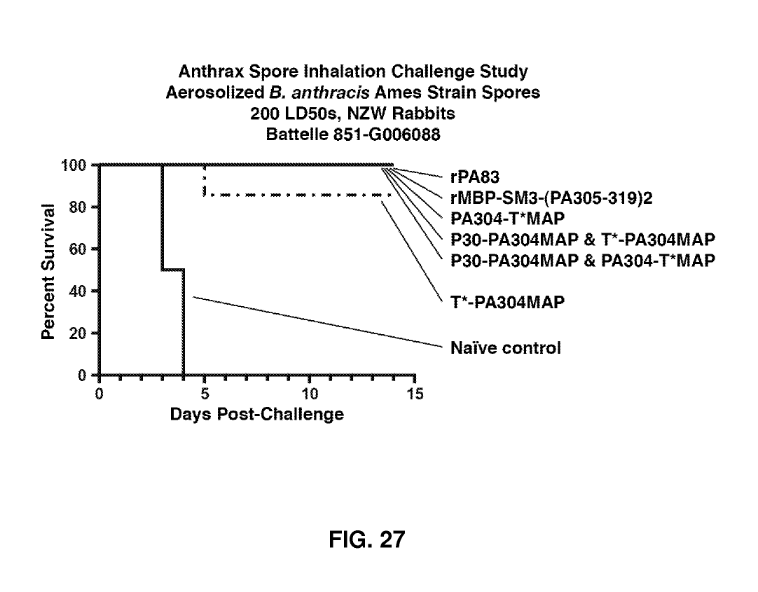

Rabbit survival post-challenge is presented in FIG. 27. The target challenge dose of aerosolized spores was successfully delivered (197 LD50s +/−38). All naive control animals died promptly (6/6), and all rPA83-immune (positive control) animals survived (7/7) as expected. All LND-containing immunogens elicited robust protective immunity against aerosolized B. anthracis Ames strain spores. The recombinant tandem repeat construct protected 7/7 rabbits, and the LND-targeted MAP peptide immunogens protected 27/28 rabbits.

The foregoing data demonstrate that a multiple antigenic peptide (MAP) comprised of 4 copies per molecule of the LND from PA, or recombinant proteins expressing the LND, can elicit humoral immunity in rabbits that is specific for the 2β2-2β3 loop of PA, and which demonstrates potent neutralization of LeTx in vitro. It is further shown that an antibody specific for the LND is not induced in rabbits immunized with full length PA and appears to not be induced in non-human primates and in humans vaccinated with the current AVA vaccine. Most importantly, an epitope-specific antibody response targeting the LND of PA, elicited using either a recombinant tandem repeat immunogen or synthetic MAP peptide immunogens, can protect rabbits from a high-dose inhalation spore challenge using a fully-virulent strain of B. anthracis, a demonstration that is critical for the reduction to practice of vaccines for this bioterrorist threat.

Synthetic Peptides and Recombinant Proteins. The synthetic peptides and recombinant proteins used in the study are listed in Table 2. A four-branch MAP displaying four copies per molecule of a.a. 305-319 from PA (single letter code: GNAEVHASFFDIGGS, GenBank accession # P13423) linked at the C-terminus to the branching lysine core of the MAP was synthesized using standard F-moc chemistry. Linear synthetic peptides used for the analysis of peptide inhibition include a.a. 305-319 from PA and an irrelevant peptide consisting of a.a. 1-16 from the Aβ42 peptide deposited pathologically in Alzheimer's disease (single letter code: DAEFRHDSGYEVHHQK). All synthetic peptides were synthesized commercially (Sigma-Genosys, the Woodlands, Tex.). Two recombinant proteins were used for assessing 305-319 peptide-specific immunity by ELISA. Both recombinant proteins were molecularly constructed, expressed and purified. The recombinants display two tandemly repeated copies of either a.a. 299-327 (single letter code: HTSEVHGNAEVHASFFDIGGSVSAGFSNS) or a.a. 305-319 (single letter code: GNAEVHASFFDIGGS) from PA linked to maltose binding protein. The DNA sequences encoding the proteins were validated with automated dideoxy sequencing of the sense and antisense DNA and the purified proteins were found to be in excess of 90% pure by SDS-PAGE analysis.

TABLE 2
Synthetic Peptides and Recombinant Proteins
Synthetic Peptides

| | |
|---|---|
| MAP peptide^{a} | 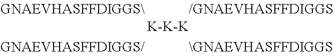 |
| PA 305-319 | GNAEVHASFFDIGGS |
| peptide | |
| Irrelevant | DAEFRHDSGYEVHHQK |
| peptide^{b} | |

Recombinant Proteins

| | |
|---|---|
| MBP-PA | MBP-(GNAEVHASFFDIGGS)₂ |
| 305-319^{c} | |
| MBP-PA | MBP- |
| 299-327^{c} | (HTSEVHGNAEVHASFFDIGGSVSAGFSNS)₂ |

^{a}The MAP peptide is comprised of four PA 305-319 peptide arms extending from α- and ε-amino groups of a branching lysine core indicated by K-K-K.
^{b}The irrelevant peptide sequence is from the Ab 1-16 peptide derived from amyloid precursor protein.
^{c}The recombinant proteins are comprised of maltose binding protein (MBP) expressed as fusions with two tandem-copies of the peptide sequences shown.

Cell Lines. RAW264.7 is a mouse macrophage cell line derived from Abelson murine leukemia virus-induced tumor in BALB/c mice (ATCC, Manassas, Va.).

Immunization of Rabbits and Sample Collection. Eight to twelve week old female C57BL/6, SJL, C3H, BALB/c or B6C3F1 (C57BL/6×C3H) mice (Jackson Laboratory, Bar Harbor, Me.) were used for all mouse experiments. For mouse antibody experiments, mice were immunized s.c. four times at two week intervals with 50 μg of PA (PA83, List Biological Laboratories, Inc., Campbell, Calif.) or 12 nanomoles of a linear T-B peptide or the P30 peptide alone mixed with 10 μg of Quil A adjuvant. Female New Zealand White (NZW) rabbits were immunized on day 0 with 500-250 μg of the MAP peptide in an emulsion with complete Freund's adjuvant (CFA) (Covance Research Products, Denver, Pa.). Rabbits were then boosted at two week intervals with 125 μg of the MAP peptide in an emulsion with incomplete Freund's adjuvant (IFA) (Sigma Biochemicals, St. Louis, Mo.). Serum samples were collected prior to the first immunization (day 0), 10 days after each booster immunization, and for some rabbits, approximately 2.5 months after the final booster immunization. Two PA-immune control rabbits were immunized with 250 μg of soluble PA in an emulsion with CFA (PA83, List Biological Laboratories, Inc., Campbell, Calif.) and then boosted 4 times at two week intervals with 125 μg of PA in IFA. Serum was obtained from the control PA-immune rabbits approximately 10 days after the fifth immunization. In two additional experiments, 7 rabbits were immunized with 250 μg of soluble PA in an emulsion with CFA and then boosted two weeks later with 125 μg of PA in IFA. Separately, 4 rabbits were immunized with 250 μg of PA using Alhydrogel adjuvant (Brenntag Biosector, Denmark) and boosted twice at two week intervals with 125 μg of PA in Alhydrogel. In both experiments, serum was obtained 10 days after the final booster immunization for analysis. Rabbits inoculated with recombinant adeno-associated vectors expressing PA63 are described in Liu, T. H. et al. (2009), Genetic vaccines for anthrax based on recombinant adeno-associated virus vectors, Mol Ther 17:373-379. All animal procedures were approved by the Institutional Animal Care and Use Committee and were performed in facilities accredited by the Association for the Assessment and Accreditation of Laboratory Animal Care, International.

T cell proliferation assay. Mice were immunized subcutaneously at the base of the tail with 12 nanomoles of the 305-319 or P30 peptide in an emulsion with CFA. Seven days after priming, para-aortic and inguinal lymph nodes were aseptically removed, and single cell suspensions were prepared. Pooled lymph node cells (LNCs), representing four to five mice per group, were cultured with either the 305-319, P30 or an irrelevant peptide, or PA in 96 well microtiter plates (Costar, Cambridge, Mass.) at 4×10⁵ cells/well in AIM V media supplemented with 3% FBS, 2 mM L-glutamine and 50×10⁻⁶ M 2-ME. The proliferative response was assessed by measuring the incorporation of 1 μCi/well of [³H]thymidine during the final 16 hours of a 4-day culture. The stimulation index (SI) for each replicate was calculated by dividing the geometric mean cpm in the presence of test antigen by the geometric mean cpm in the presence of media alone.

ELISA Analysis. Individual rabbit antisera were analyzed in duplicate by ELISA, as described by Oscherwitz, J. et al., (2006) Low-dose intraperitoneal Freund's adjuvant: toxicity and immunogenicity in mice using an immunogen targeting amyloid-beta peptide, Vaccine 24:3018-3025. For analysis of antibodies specific for the PA, wells of microtiter plates (Immulon 2, Thermo Labsystems, Franklin Mass.) were coated overnight at 4° C. with 100 ng of PA (PA83, List Biological Laboratories, Inc., Campbell, Calif.) in a 0.05 M carbonate buffer pH 9.5. For analysis of anti-peptide binding, wells were coated with 100 ng of a recombinant protein displaying two tandemly repeated copies of either a.a. 299-327 or a.a. 305-319 from PA, both expressed as fusions with maltose binding protein. Bound antibody was detected with secondary biotinylated antibody specific for rabbit IgG (Southern Biotechnology, Birmingham, Ala.) followed by streptavidin-alkaline phosphatase and 4-nitrophenylphosphate (Roche, Indianapolis, Ind.). Absorbance at 405 nm minus absorbance at 650 nm was determined using an ELISA reader (Emax microplate reader, Molecular Devices, Menlo Park, Calif.). Antibody titers were determined from serial two-fold dilutions of individual rabbit serum and represent the reciprocal dilution at the EC50 established using nonlinear regression to fit a variable slope sigmoidal equation to the serial dilution data using Prism 5.0 (GraphPad Software, Inc., San Diego, Calif.). The lower limit of assay detection was 16.

Avidity assays were based on ELISA quantitation of bound antibody in the presence or absence of chaotrope as described by Anttila et al. (Avidity of IgG for Streptococcus pneumoniae type 6B and 23F polysaccharides in infants primed with pneumococcal conjugates and boosted with polysaccharide or conjugate vaccines, J Infect Dis 177:1614-1621) with slight modification, as per Albrecht, M. T. et al. (2007), Human monoclonal antibodies against anthrax lethal factor and protective antigen act independently to protect against Bacillus anthracis infection and enhance endogenous immunity to anthrax, Infect Immun 75:5425-5433. Briefly, after initial exploratory studies with several chaotropes at a range of concentrations, 2 M NH₄SCN was selected based on the maximal discrimination of antibody binding with this chaotrope. For the chaotrope titration, duplicate plates were coated with PA and ELISA procedures were followed as described above in the methods section. After antisera incubation, plates were washed in wash buffer and chaotrope plates received 100 microliters of 2 M NH₄SCN (Sigma Biochemicals, St. Louis, Mo.) and were incubated for 30 minutes at room temperature. Non-chaotrope plates were handled in parallel but received only wash buffer in place of chaotrope. After the chaotrope step, both plates were washed and processed as usual for the remainder of the ELISA. The avidity index, which represents the fraction of bound antibody resistant to chaotrope, was determined for each serum, and is defined as the EC50 antibody titer in the presence of a chaotrope elution, divided by the EC50 titer without chaotrope elution, multiplied by 100. EC50 titers were determined as described in the ELISA methods section.

Antibody Affinity. For determination of peptide IC₅₀, serial two-fold dilutions starting at 256 μM (2×) in dilution solution (PBS, 2% BSA, 0.05% Tween 20) were performed with either the P30-PA304-319 peptide or the P30 peptide. Experimental serum was added to the diluted peptides in duplicate at a predetermined dilution of one-half the reciprocal EC₅₀ titer (2×). The serum/peptide mixtures were allowed to equilibrate at room temperature for 2 hours and then overnight at 4° C. The following day, the serum/peptide mixtures were analyzed in the standard ELISA as described above and peptide IC₅₀ were determined for each serum using nonlinear regression to fit a variable slope sigmoidal equation to the serial dilution data using Prism 5.0 (GraphPad Software, Inc., San Diego, Calif.). Peptide IC₅₀s were considered to represent good approximations of the antibody dissociation constant (Kd).

Toxin Neutralization Assays. The ability of antibody to block LeTx action in vitro was assessed in the toxin neutralization assay (TNA) using the RAW264.7 cell line (ATCC, Manassas, Va.) essentially as described. Briefly, cells were grown in culture in Dulbecco's modified Eagles medium (DMEM) with 10% fetal bovine serum, penicillin-streptomycin and 50 μM 2ME (complete medium) in a humidified 6.5% CO₂ incubator. Complete medium was used for dilution of all assay reagents. For each experiment, cells were harvested using 3 mM EDTA, washed with DMEM and plated at 30×10³ cells/well in 96-well flat bottom plates for overnight culture (Costar 3596, Corning Inc., Corning, N.Y.). The following day, heat-denatured rabbit antisera in duplicate was serially diluted in polypropylene round-bottom 96 well plates in a final volume of 50 microliters per well. LeTx reagent containing PA83 and LF in complete medium was prepared at a twice-final (2×) concentration, with the final concentration representing 2.5 to 3.5 multiples of the amount needed to kill 50% of the RAW264 cells. Each TNA assay was validated by a contemporaneous PA titration. For each neutralization assay, 110 ng/ml PA83 was used along with 150 ng of LF. The diluted rabbit antiserum was added to the LeTx and the mixture was incubated for 30 minutes before transferring to the RAW264 cells in exchange for the pre-existing medium. Following a 4 hour incubation, 20 microliters of MTS reagent was added to each well (CellTiter96 AQ, Promega Corp., Madison, Wis.), and after an additional 2 hour incubation, the absorbance at 405 nm minus absorbance at 650 nm was determined for each plate using a Vmax plate reader. Neutralization ED50 (effective dilution at which 50% of cells are protected from cytotoxicity) titers were determined from serial two-fold dilutions of individual rabbit serum and represent the reciprocal dilution at the EC50 established using nonlinear regression to fit a variable slope sigmoidal equation to the serial dilution data using Prism 5.0 (GraphPad Software, Inc., San Diego, Calif.). The lower limit of assay detection was about 16. For the analysis of peptide inhibition of TNA, experimental serum samples were pre-incubated with 20 μM peptide for 30 minutes at room temperature prior to analysis in the TNA.

Statistical Analysis. For determination of ELISA EC50 titers and TNA ED50 titers, four parameter logistic regression was used to fit variable slope sigmoidal equations to the serial dilution data. The Student's t-test was used to compare antibody affinity between cohorts of rabbits immunized with MAP peptides containing the P30 helper epitope or with no helper epitope. A P value of <0.05 was considered statistically significant. Spearman's rho correlation was used to calculate correlations between IC₅₀ data and antibody titers with normalized TNA levels. All statistical analysis was performed using GraphPad Prism software version 5.0 (GraphPad Software, Inc., San Diego, Calif.).

One alternative immunogen was reported by Yin, Y. et al. (2008) Chimeric hepatitis B virus core particles carrying an epitope of anthrax protective antigen induce protective immunity against Bacillus anthracis, Vaccine 26(46): 5814-5821 (hereinafter Yin), where sequences from the 2β2-2β3 loop were inserted into the major immunodominant region of hepatitis B core protein and the recombinant fusion protein was expressed as virus like particles. Serum from guinea pigs immunized with the virus like particles demonstrated LeTx neutralizing activity and were partially protected from a subcutaneous challenge with B. anthracis.

The present data can be contrasted with the data reported by Yin. The Yin document describes the use of a recombinant protein as an immunogen, being a fusion protein of the hepatitis C virus core protein fused to a single copy of the epitope, where the epitope is inserted within the hepatitis C virus core protein. The Yin fusion protein forms virus-like particles (VLPs) and is expressed in E. coli. Mice and Hartley Guinea pigs were immunized with the fusion protein using a strong adjuvant in the mice experiments (CFA/IFA) and either a weak (alhydrogel) or no adjuvant was used in Guinea pig experiments. The fusion protein immunogenicity resulted in antibody titers (ELISA) results of approximately 500 for EC50. The toxin neutralization assay (TNA) was less reliable, where 40 nanograms PA and 20 nanograms LF were used. Also, the “endpoint” titers were used to describe in vitro results, which is non-standard in current anthrax literature. The toxin neutralization results were also relatively low, being about 50-100 for EC50 titers. These results are only about 1% to 2.5% of the PA controls. These experiments result in very low doses of toxin in vitro with very low titer results, especially considering how little toxin is present in the assay.

The Yin document's in vivo protection testing was furthermore done subcutaneously, not by inhalation. The protection assay results show only 4/7 (57%) were protected with no adjuvant and 3/7 (37%) were protected with alhydrogel. PA (positive control) protected 2/3 GPs (66%) and irrelevant immune protected 1/8 (12.5%). The challenge isolate was IVDC40048. In general, these are very small challenge numbers. The protection also does not appear appreciable or statistically significant (comparison between irrelevant group and other groups does not appear to be significant, although no statistics are presented). The Guinea pig results also present somewhat of an unpredictable challenge model for anthrax, although this model has been used in early anthrax work. The virulence of the Bacillus anthracis challenge strain is also an unknown. The test groups also do not appear to have been randomized.

In contrast, the present experiments using LND variants demonstrate specific and robust immunization and protection. The present experiments describe LND immunogens including recombinant proteins, fusion proteins with E. coli maltose binding protein MBP) fused to tandem repeat B cell epitopes, synthetic peptides, including completely synthetic linear peptides and multiple antigenic peptide (MAP) constructs. Animals used include mice and rabbits, where the rabbit is considered one of two (along with non-human primates) accepted animal models for testing potential vaccines for anthrax. Adjuvants include the strong adjuvants used in the mouse and rabbit work and some weak adjuvants used in rabbit work; e.g., alhydrogel and alhydrogel combined with CpG, QS-21 and MPL. Immunogenicity of the LND variants produced antibody titers about 5,000-50,000 for EC50. The toxin neutralization assay data used 110 nanograms of PA and 150 nanograms of LF, and used EC50 titers which are the recognized standard in the anthrax literature. The present experiments also demonstrate very high TNA results, with a range of about 250 to about 20,000, where these results are from about 50% to about 2,000% of control PA neutralization.

The present in vivo protection testing included inhalation protection assay results. The data indicate the LND immunization protected 34/35 rabbits overall (97%), with most groups tested being 100% protected, where only one group immunized with a synthetic peptide lost a single rabbit. These experiments used a well established virulent Ames strain of anthrax of known high virulence. Inhalation challenge with aerosolized spores is the known gold standard challenge model. Thus, the present data arise from a very robust challenge and provide excellent protection (almost complete protection overall, complete in all but one group). The present experiments and data were also performed at a 3rd party site in blinded and group randomized fashion.

As a result, the present disclosure deals with vaccines that protect against inhalation anthrax. The Yin document deals with a similar epitope, but demonstrates lower immunogenicity and lower toxin neutralization. Notably, the Yin document also does not disclose any data demonstrating protection from inhalation anthrax, which is the relevant challenge to demonstrate efficacy.

The following listing of embodiments provides additional examples of the present disclosure:

Embodiment 1 provides a composition or a method of making a vaccine comprising a polypeptide containing the Bacillus anthracis protective antigen epitope comprised of residues 308-318 of SEQ ID NO: 1 or functional variants thereof.

Embodiment 2 is a composition of embodiment 1 wherein contiguous N- or C-terminal residues are included derived from Bacillus anthracis protective antigen 299-327 of SEQ ID NO: 2.

Embodiment 3 is a composition of embodiment 2 comprising the sequence of SEQ ID NO: 3.

Embodiment 4 is a composition of embodiment 1 wherein the composition comprises linked polypeptide sequences that stimulate helper T lymphocyte responses.

Embodiment 5 is a composition of embodiment 4 wherein the helper T lymphocyte epitopes are selected from the group consisting of Clostridium tetani toxin 947-967, Clostridium tetani 830-844, Plasmodium falciparum circumsporozoite protein 326-345, Shistosoma mansoni 38 kDa Soluble Egg Antigen 235-249, measles virus fusion protein 288-303.

Embodiment 6 is a composition of embodiment 1 wherein the composition further comprises N- and C-terminal spacer and crosslinking residues.

Embodiment 7 is a composition of embodiment 1 wherein the composition is a synthetic peptide.

Embodiment 8 is a composition of embodiment 1 wherein the composition is a recombinant protein.

Embodiment 9 is a composition of embodiment 7 wherein the polypeptide is linear.

Embodiment 10 is a composition of embodiment 7 wherein the polypeptide is cyclized.

Embodiment 11 is a composition of embodiment 7 wherein the composition is a branching structure.

Embodiment 12 is a composition of embodiment 11 wherein the composition is a multiple antigen peptide system.

Embodiment 13 is a composition of embodiment 1 wherein a polypeptide segment containing SEQ ID NO: 1 is repeated.

Embodiment 14 is a composition of embodiment 8 wherein the recombinant protein is a fusion protein.

Embodiment 15 is a composition of embodiment 14 wherein the fusion protein contains Escherichia coli maltose binding protein or derivatives thereof.

Embodiment 16 is a composition of embodiment 8 wherein the polypeptide is encoded by DNA and expressed in the vaccinated individual.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. An immunogen comprising:
a loop neutralizing determinant peptide, the peptide comprising amino acids 304-319 or amino acids 305-319 of Bacillus anthracis protective antigen or a functional variant thereof; and
a helper T-cell epitope coupled to the peptide.

2. The immunogen of claim 1, wherein the functional variant of the peptide comprises at least 75% identity to amino acids 304-319 or amino acids 305-319 of Bacillus anthracis protective antigen and provides at least 50% binding activity to an antibody to amino acids 304-319 or amino acids 305-319 of Bacillus anthracis protective antigen.

3. The immunogen of claim 1, wherein the helper T-cell epitope is only coupled to the N-terminus or to the C-terminus of the peptide.

4. The immunogen of claim 1, wherein the helper T-cell epitope is only coupled to the N-terminus of the peptide.

5. The immunogen of claim 1, wherein the loop neutralizing determinant peptide comprises a tandem repeat of the peptide.

6. The immunogen of claim 1, wherein the helper T-cell epitope is selected from the group consisting of P30 from Clostridium tetani toxin 947-967, Clostridium tetani toxin 830-844, Plasmodium falciparum circumsporozoite protein 326-345, Shistosoma mansoni 38 kDa soluble egg antigen 235-249, measles virus fusion protein 288-303, and combinations thereof.

7. The immunogen of claim 1, wherein the helper T-cell epitope is P30 from Clostridium tetani toxin 947-967 and the C-terminus of the P30 is coupled to the N-terminus of the loop neutralizing determinant peptide.

8. The immunogen of claim 1, wherein the helper T-cell epitope is Shistosoma mansoni 38 kDa soluble egg antigen 235-249 and three copies of the helper T-cell epitope are covalently linked to two copies of the peptide comprising amino acids 305-319 of Bacillus anthracis protective antigen or a functional variant thereof.

9. The immunogen of claim 1, wherein the loop neutralizing determinant peptide and the helper T-cell epitope are expressed as a recombinant polypeptide.

10. The immunogen of claim 9, wherein the recombinant polypeptide further comprising an affinity tag.

11. The immunogen of claim 10, wherein the affinity tag is Escherichia coli maltose binding protein.

12. The immunogen of claim 11, wherein the recombinant protein comprises maltose binding protein fused with three tandem copies of a helper T cell epitope from Shistosoma mansoni soluble egg antigen P38 235-249 and two tandem copies of the peptide comprising amino acids 305-319 of Bacillus anthracis protective antigen or a functional variant thereof.

13. The immunogen of claim 1, further comprising at least one pharmacologically acceptable excipient.

14. The immunogen of claim 1, further comprising an adjuvant.

15. The immunogen of claim 14, wherein the adjuvant is selected from the group consisting of alhydrogel with monophosphoryl lipid A (MPL), Quil A, complete Freund's, incomplete Freund's adjuvant, and combinations thereof.

16. The immunogen of claim 1, wherein at least a portion of the immunogen is cyclized.

17. An immunogen comprising a multiple antigenic peptide comprising a plurality of segments, each segment comprising a loop neutralizing determinant peptide comprising amino acids 304-319 or amino acids 305-319 of Bacillus anthracis protective antigen or a functional variant thereof.

18. The immunogen of claim 17, wherein the functional variant of the loop neutralizing determinant peptide comprises at least 75% identity to amino acids 304-319 or amino acids 305-319 of Bacillus anthracis protective antigen and provides at least 50% binding activity to an antibody to amino acids 304-319 or amino acids 305-319 of Bacillus anthracis protective antigen.

19. The immunogen of claim 17, wherein the segments are configured in a branching manner.

20. The immunogen of claim 17, wherein the segments are linked via α- and/or ε-amines of a branching lysine core.

21. The immunogen of claim 17, wherein the segments are synthesized as linear peptides and subsequently conjugated to a branching lysine core.

22. The immunogen of claim 17, wherein the segments are synthesized as linear peptides and subsequently conjugated to a dendrimeric core.

23. The immunogen of claim 17 comprising four segments.

24. The immunogen of claim 17, wherein at least one segment further comprises a helper T-cell epitope coupled to the loop neutralizing determinant peptide.

25. The immunogen of claim 22, wherein the helper T-cell epitope is only coupled to the N-terminus or to the C-terminus of the peptide.

26. The immunogen of claim 22, wherein the helper T-cell epitope is only coupled to the N-terminus of the peptide.

27. The immunogen of claim 22, wherein the helper T-cell epitope is selected from the group consisting of P30 from Clostridium tetani toxin 947-967, Clostridium tetani toxin 830-844, Plasmodium falciparum circumsporozoite protein 326-345, Shistosoma mansoni 38 kDa soluble egg antigen 235-249, measles virus fusion protein 288-303, and combinations thereof.

28. The immunogen of claim 22, wherein the C-terminus of the helper T-cell epitope is coupled to the N-terminus of the loop neutralizing determinant peptide.

29. The immunogen of claim 22, wherein the helper T-cell epitope is P30 from Clostridium tetani toxin 947-967 and the C-terminus of the P30 is coupled to the N-terminus of the loop neutralizing determinant peptide.

30. The immunogen of claim 22, wherein the helper T-cell epitope is P30 from Clostridium tetani toxin 947-967 or Plasmodium falciparum circumsporozoite protein 326-345 and the C-terminus of the helper T-cell epitope is coupled to the N-terminus of the loop neutralizing determinant peptide.

31. The immunogen of claim 22 comprising four segments, wherein each segment comprises a Plasmodium falciparum circumsporozoite protein 326-345 coupled to a loop neutralizing determinant peptide

32. The immunogen of claim 17, wherein the B cell epitope segment in the peptide or protein is cyclized.

33. The immunogen of claim 17, further comprising at least one pharmacologically acceptable excipient.

34. The immunogen of claim 17, further comprising an adjuvant.

35. The immunogen of claim 17, wherein the adjuvant is selected from the group consisting of alhydrogel with monophosphoryl lipid A (MPL), Quil A, complete Freund's, incomplete Freund's adjuvant, and combinations thereof.

36. The immunogen of claim 17, wherein at least a portion of the immunogen is cyclized.
